(19) United States
(12) Patent Application Publication
(10) Pub. No.: US 2026/0218307 A1
(43) Pub. Date: Jul. 30, 2026

(54) DIAGNOSIS OF SKIN CONDITIONS IN VETERINARY AND HUMAN PATIENTS

(71) Applicants: UNIVERSITY OF MASSACHUSETTS, Westborough, MA (US); TRUSTEES OF TUFTS COLLEGE, Medford, MA (US)

(72) Inventors: Jillian M. RICHMOND; Jadesola OLAYINKA, New York, NY (US); Ramon M. ALMELA, Shrewsbury, MS (US)

(21) Appl. No.: 19/141,960

(22) PCT Filed: Oct. 16, 2023
(86) PCT No.: PCT/US2023/035230

§ 371 Date: Jun. 20, 2025

Related U.S. Application Data
(60) Provisional application No. 63/478,900, filed on Jan. 6, 2023.

(51) Int. Cl. C12Q 1/6886; A61K 45/06; C12Q 1/6837

(52) U.S. Cl. CPC C12Q 1/6886; A61K 45/06; C12Q 1/6837; C12Q 2600/16; G01N 2800/52

(57) ABSTRACT

Described herein are methods for diagnosing and treating mycosis fungoides (MF) and cutaneous T cell lymphoma (CTCL), e.g., for making a differential diagnosis of CTCL/MF versus other skin conditions.

### CLAIM OF PRIORITY

This application claims the benefit of U.S. Provisional Patent Application Ser. No. 63/478,900, filed on Jan. 6, 2023. The entire contents of the foregoing are hereby incorporated by reference.

### TECHNICAL FIELD

Described herein are methods for diagnosing and treating mycosis fungoides (MF) and cutaneous T cell lymphoma (CTCL), e.g., for making a differential diagnosis of CTCL/MF versus other skin conditions.¹

### BACKGROUND

Cutaneous T cell lymphomas (CTCL) are a heterogenous group of non-Hodgkin's lymphomas characterized by the proliferation of neoplastic T-lymphocytes in the skin.

### SUMMARY

Mycosis Fungoides (MF) is the most common subtype of Cutaneous T Cell Lymphoma (CTCL), a cancer of T cells that migrate to the skin. Clinically, the lesions caused by MF can look very different and mimic other skin conditions such as vitiligo, atopic dermatitis and others, particularly in skin of color patients. Early and accurate diagnosis is vital to improve prognosis. Described herein are methods for diagnosis of CTCL/MF, e.g., for making a differential diagnosis of CTCL/MF versus other skin conditions.

Thus, provided herein are methods comprising: obtaining a sample comprising skin from a subject; and determining a level of expression of one, two, three, or all four of cathepsin W (CTSW), T Cell Receptor Associated Transmembrane Adaptor 1 (TRAT1), killer cell lectin like receptor K1 (KLRK1; also known as NKG2D), and/or XCL1/XCL2 (lymphotactin) in the sample.

Also provided herein are methods of diagnosing a subject as having cutaneous T cell lymphoma. The methods include obtaining a sample comprising skin from a subject; determining a level of expression of one, two, three, or all four of cathepsin W (CTSW), T Cell Receptor Associated Transmembrane Adaptor 1 (TRAT1), and killer cell lectin like receptor K1 (KLRK1), and/or XCL1/XCL2 (lymphotactin) in the sample; comparing the level of expression of CTSW, TRAT1, KLRK1 and/or XCL1/XCL2 in the sample to a reference level, and identifying the subject as having CTCL wherein a level of expression of CTSW, TRAT1, KLRK1 and/or XCL1/XCL2 at or above the reference level indicates that the subject.

In some embodiments, the sample comprises skin from a lesion, e.g., a nodule, tumor, plaque, ulcerated, or rashy patch of skin that is suspected of being CTCL. In some embodiments, the subject is a mammal. In some embodiments, the subject is a human, e.g., a human subject having skin of color. In some embodiments, the subject has Fitzpatrick phototype IV, V, or VI skin.

In some embodiments, the subject is a canine, feline, or equine.

In some embodiments, the methods further comprise selecting and optionally administering a treatment for CTCL to a subject identified as having CTCL. In some embodiments, the treatment comprises a skin-directed therapy, optionally topical steroids, involved-site radiation therapy (RT), total skin electron-beam therapy (TSEBT), or phototherapy/photopheresis, or systemic therapies, optionally immunotherapies (retinoids, interferon, chimeric antigen receptor T cells, or forodesine), small molecules (histone deacetylase inhibitors), antibodies (optionally anti-CCR4, anti-programmed cell death protein 1, anti-CD47, or anti-CD30), chemotherapy, or allogeneic haematopoietic stem cell transplantation.

Also provided herein are kits comprising reagents for detecting the biomarkers described herein, e.g., kits comprising probes or primers that bind to the biomarkers described herein, and one or more control sequences, e.g., a housekeeping gene. In some embodiments the kits comprise microarrays. In some embodiments, the kits comprise fewer than 10 or 20 probes or primers.

Unless otherwise defined, all technical and scientific terms used herein have the same meaning as commonly understood by one of ordinary skill in the art to which this invention belongs. Methods and materials are described herein for use in the present invention; other, suitable methods and materials known in the art can also be used. The materials, methods, and examples are illustrative only and not intended to be limiting. All publications, patent applications, patents, sequences, database entries, and other references mentioned herein are incorporated by reference in their entirety. In case of conflict, the present specification, including definitions, will control.

Other features and advantages of the invention will be apparent from the following detailed description and figures, and from the claims.

### DESCRIPTION OF DRAWINGS

FIG. 1. Clinical presentations of epitheliotropic lymphoma in dogs. Top row left to right: widely disseminated lesions with hair loss associated with severe scale-crusting in an English bulldog and a retriever mix. Mild crusting with depigmentation, loss of nose cobblestone appearance and erythema on the muzzle of a beagle mix. Middle row left to right: alopecia, erythema and crusting on the nasal planum and hind legs of a schnauzer mix; depigmentation, loss of nose cobblestone appearance and erythema on the muzzle of a beagle mix with footpad involvement with crusting and ulcerations. Note the depigmentation on the nasal planum and muzzle, which can be mistaken for other immune-mediated processes. Bottom row left to right: multifocal lesions with erythema and crusting and without notable hair loss in a pitbull mix. Erythematous alopecia patch and hyperkeratotic plaque lesions on the dorsum of a shepherd mix. Please note that breed is not associated with a particular presentation of the disease.

FIGS. 2A-D. Gene expression in epitheliotropic lymphoma (EL) versus healthy margin controls reveals CD8+ cytotoxic T cell signatures. A. Sample H&E photomicrographs of healthy and EL tissue. B. Volcano plot of differentially expressed genes (DEGs) between healthy and EL. C. Heatmap of DEGs generated with Rosalind software. D. Principal component analysis (PCA) of cases versus healthy margins generated with ClusVis software. (n=6 EL and 5 healthy controls, Padj<0.05 considered significant).

FIGS. 3A-D. Gene expression in EL versus other interface dermatitis conditions reveals CTSW and TRAT1 can distinguish between malignancy and other immune mediated processes. A. Volcano plot of EL versus other interface dermatitis conditions including cutaneous lupus erythematosus, pemphigus, and erythema multiforme spectrum disorders. B. Heatmap generated in Rosalind software. CTSW gene highlighted by a rectangle. C. Raw counts of the top DEGs between EL and ID (one-way ANOVA with Tukey's post tests significant as indicated). D. Receiver operator characteristic (ROC) curves of CTSW, TRAT1 and KLRK1. (n=6 EL and 31 other interface dermatitis conditions; Padj<0.05 considered significant.)

FIGS. 4A-E. Validation cohort confirms CTSW and TRAT1 as potential biomarkers for distinguishing EL and ID. A. Volcano plot of EL versus other interface dermatitis conditions. B. Heatmap and Gene Set Analysis generated in Rosalind software. C. Raw counts of the top DEGs between EL and ID (one-way ANOVA with Tukey's post tests significant as indicated). D. Receiver operator characteristic (ROC) curves of CTSW, TRAT1, and XCL1/XCL2. E. Multi-ROC curve combining CTSW, TRAT1 and KLRK1 normalized counts for distinguishing EL from ID in the discovery and validation cohorts (discovery cohort: n=6 EL and 31 other interface dermatitis conditions; validation cohort: n=6 EL and 9 other interface dermatitis conditions; Padj<0.05 considered significant.)

FIGS. 5A-D. Comparative transcriptomics of human MF and canine EL versus ID DEGs. A. Geo2R reanalysis of GSE143382 human MF dataset. DEGs from dermatitis vs early MF were used as they represent the closest clinical match. B. DEG overlap between the canine validation cohort (canine IO panel) and human dataset (myeloid v2 panel) using the 327 common denominator genes from the respective NanoString probesets. C. Comparison of previously published up- and downregulated genes in canine and human datasets. D. Analysis of TOX vs TRAF1, which was used to distinguish human MF from ID, or CTSW vs TRAT1 in canine samples.

FIG. 6. Immunohistochemical analysis of CTSW using a human/murine reactive antibody.

FIGS. 7A-B. Immunohistochemical analysis of CD244 using a human/murine reactive antibody. A. EL. B. Discoid lupus erythematosus (DLE).

FIGS. 8A-B. qPCR of CTSW does not work reliably on FFPE RNA. qPCR was performed on RNA from FFPE skin (A) or frozen lymph node aspirates (B). CTSW set 2 may be used to detect transcript in frozen but not FFPE RNA samples.

FIG. 9. Truncated microarray diagnostic tool works reliably on FFPE RNA. A truncated microarray diagnostic tool was developed using four biomarkers of interest (CTSW, TRAT1, KLRK1, and Loc490356/lymphotactin/XCL1/XCL2) and five housekeeping gene controls (B2M, RPL13A, CCZ1, HPRT1, and GAPDH). This tool was able to sensitively and specifically detect EL in six dogs apart from six ID cases (RNA was pulled from both discovery and validation cohort samples).

### DETAILED DESCRIPTION

The World Health Organization-European Organization for Research and Treatment of Cancer (WHO-EORTC) created a classification consensus recognizing 19 distinct subtypes of CTCL.¹ The most common subtype is mycosis fungoides (MF), which is known for its progression of three stages: patches, plaques, and tumors²—of which their mushroom-like appearance inspires the name. Patients can present with all three stages simultaneously, or may experience one stage for years. Other important subtypes of CTCL include Sezary syndrome, which is the leukemic variant of CTCL characterized by malignant Sezary cells in the plasma, and primary cutaneous CD30+ lymphoproliferative disorders, which include lymphomatoid papulosis and CD30+ primary cutaneous anaplastic large cell lymphoma.⁴⁰ Along with MF, these subtypes have indolent clinical behavior, while Sézary syndrome has aggressive clinical behavior.¹

In humans, CTCLs often present with lesions such as red patches, plaques, nodules, ulcerations, or other skin rashes, and can evolve into cutaneous tumors and/or progress to visceral involvement. In the United States, the overall annual age-adjusted incidence of CTCL was 6.4 per million persons over the time period of 1973 to 2002, with an annual incidence increase of 2.9×10⁻⁶. ³ Incidence is greater in males than in females, and in Black people than in White people.

MF has a chronic, relapsing course, and patients often try several therapies to control the course of their disease⁴³. The prognosis of MF depends highly upon the stage at which a patient is diagnosed: for stage IA in patients who respond well to therapy, their overall survival rate is similar to age matched controls. However, for stage IIB to stage III, patients have a median survival rate of 3.2-6 years⁴⁴. Stage IV, in which patients develop extracutaneous symptoms, patients have a survival rate of less than 1.5 years. Historically, CTCL has been considered an “old white man's disease,” primarily affecting Caucasian men over age 60⁴⁵. However, CTCL affects African Americans (AA) and other skin of color (SOC) patients, with up to 70% female bias and a younger age at presentation for the MF subtype (mean age 44 years)⁴⁶⁻⁴⁹. MF disproportionately affects SOC patients: patients are often diagnosed at a later stage, making treatment more difficult and less effective at controlling disease⁵⁰⁻⁵³. Thus, SOC patients have worse prognoses than white patients. Further, SOC patients are less likely to be referred to extracorporeal photopheresis, a standard treatment for CTCL⁵⁴. A better understanding of MF pathogenesis in SOC patients is needed both for early identification and effective treatment, as many treatments to date have been tested and developed in predominantly white patient populations⁵⁵.

In addition to humans, other mammals including dogs, cats, and horses can also develop cutaneous lymphomas, including T and B cells lymphomas.^{56,57,64-66} In canine cutaneous epitheliotropic lymphoma (EL), neoplastic lymphocytes also infiltrate the skin and mucosa.⁴ Canine epitheliotropic T cell lymphoma (T-EL) has different subtypes when described with the same standards as humans, including MF, Sézary syndrome, and pagetoid reticulosis⁵. While CD4+ helper T-cells predominantly drive disease in human CTCL, canine EL is predominantly a disease of CD8+ cytotoxic T-cells.⁵ The disease progression in both human CTCL and canine EL can be very similar. Human CTCL usually involves progression from patch stage to plaque stage to tumor stage, whereas in canine patients with MF, the variety of lesions can present at any time during disease development and each lesion type does not necessarily represent a progressive disease stage. Clinical presentations of both diseases involve lesions such as exfoliative erythroderma, ulceration, depigmentation, plaques, and nodules, and both diseases are difficult to diagnose in earlier stages due to similar clinical presentation to inflammatory or benign processes⁶⁶. While CD4+ helper T-cells predominantly drive disease in human CTCL, canine T-EL is predominantly a disease of CD8+ cytotoxic T-cells⁵.

MF in SOC can mimic other conditions, potentially leading to misdiagnosis of patients and hence delayed treatment start^{49,58}. MF presentation varies widely⁴⁹: it can present as flat or raised lesions, plaques with an annular morphology, scale, or violaceous plaques, psoriasiform lesions, hyper- and/or hypo-pigmented lesions, ulcerations, or nodules. In SOC patients, depigmented MF is one of the most common presentations, which may be mistaken for vitiligo, fungal infection, or other dermatitis conditions.

Forms of CTCL that express CD8+ phenotype in humans have been reported³¹: in some rare cases, well-defined types of CTCL (such as MF) express CD8+ and have similar clinical presentation and disease prognosis as the more common CD4+ cases. Outside of these, studies suggest separate groups of more aggressive CD8+ cytotoxic CTCLs, including an epidermotropic type and a panniculitis-like subcutaneous T cell lymphoma type.

One challenge in the field is distinguishing early-stage MF in humans, particularly human subjects with skin of color, and dogs from other immune-mediated interface dermatitis conditions. The present results expand understanding of these disorders and provide biomarkers for diagnosis disease, improving prognosis and treatment response, and allows the development of targeted therapies, e.g., repurposing known drugs, for veterinary and human medicine. As shown herein, CTSW, KLRK1 and TRAT1 may be used to distinguish EL from other forms of interface dermatitis (ID) in humans and veterinary subjects in both a discovery and validation cohort. XCL1/XCL2 was also highly significant at distinguishing EL from other forms of ID in the validation cohort. These genes may be particularly important to prevent misdiagnosis in both veterinary and human patients, in order to distinguish CTCL from clinical mimickers.

CTSW is a member of the cathepsin S family that is a papain-like protease. Cathepsins have been reported to regulate cancer progression and therapeutic responses⁸. Cathepsin S is upregulated in follicular lymphoma, and an activating mutation Y132D drives lymphomagenesis through alterations in antigen presentation⁹ and a pro-tumorigenic microenvironment¹⁰. Cathepsin W is known to be expressed in human CD8+ T cells¹¹ and Natural Killer (NK) cells.^{12,13} Similarly, family member Cathepsin G is 2-fold upregulated in early disease stages (IA/IB).¹⁴ In breast cancer, increased expression of Cathepsin B and Cathepsin L is associated with poorer prognosis, greater mortality, and greater disease metastases.⁸ In colorectal cancer, elevated Cathepsin B and Cathepsin L are associated with increased disease metastasis and poorer prognosis¹⁵, while elevated Cathepsin S predicts both decreased survival when treated with surgery alone, with potential benefit from adjuvant 5-fluorouracil and folinic acid treatment. High Cathepsin B expression in lung, ovarian, pancreatic neuroendocrine cancers and pancreatic adenocarcinomas, is negatively correlated with survival and positively associated with recurrence, invasion, and/or tumor grade. Upregulation of Cathepsin K in osteosarcomas predicts poor prognosis and disease metastasis. Generally, it seems that the tumor microenvironment helps activate cathepsins, which in turn activate oncogenesis: mutant HRAS in mammary epithelial cells upregulates CTSB and CTSL; the HER2 oncogene drives expression of CTSB through the transcription factor myeloid zinc finger 1 (MZF1), and CTSB is a functional driver of the invasive phenotype.

TRAT1 is a 30 Kd type III transmembrane protein expressed by human T lymphocytes and natural killer (NK) cells. TRAT1 regulates T-cell receptor expression.¹⁶ It consists of an extracellular domain, transmembrane region, and cytosolic tail. TRAT1 facilitates CTLA-4 shuttling from the trans Golgi network to the T-cell surface where it stabilizes the T-cell antigen receptor and CD3 complex. TRAT1 knockdown experiments demonstrated reduced CTLA-4 mediated cytokine release as well as CTLA-4 cell surface expression and subcellular distribution. When TRAT1 is overexpressed in Jurkat T cells, there is an increase in T-cell receptor expression, however this is not the case in regular T cells.¹⁷ By contributing to the structural integrity of the TCR/CD3 complex, TRAT1 has a significant role in TCR functions, which include triggering antigen-specific T-cell responses.¹⁸ In non-blood cancers (solid tumors), TRAT1 expression is important for a good prognosis. However in T cell lymphomas, TRAT1 can be highly expressed in tumor cells. It remains unknown how this relates to prognosis.

KLRK1 is also known as NKG2D. It is a killer type lectin receptor expressed on NK cells and cytotoxic T cells.¹⁹ KLRK1 is upregulated in human peripheral T cell lymphomas.²⁰ A recent study demonstrated that benign T cells drive inflammation in MF tumors in humans²¹: an influx of CD8+ T cells following immunotherapy in CD4-driven tumors was protective. Therefore, caution should be exercised in fully characterizing the tumor (e.g. CD4 or CD8; does the tumor itself bear KLRK1, or is it expressed by the infiltrating T cells²²) to ascertain whether KLRK1 both as a biomarker and a potential treatment target is beneficial or detrimental.²¹

LOC490356 was a top DEG in our validation cohort. This locus encodes lymphotactin, also known as XCL1/XCL2. This chemokine is homeostatically expressed by NK cells and has antimicrobial activity.²³ XCL1 is downregulated in human Sezary Syndrome,^{24,25} but was expressed by T-3B cells in a case report of a patient with concurrent T and B cell cutaneous lymphomas²⁶ and by T cells in a patient with lymphoproliferative disorder.²⁷ As with KLRK1, it is unclear whether XCL1 could be protective or pathogenic in the EL setting. In murine tumor models, injection of XCL1-expressing myeloma cells in Balb/c and nude mice resulted in tumor regression.²⁸ An XCL1 fusion peptide improved tumor rejection in a mouse B16 model of melanoma via recruitment of XCR1+ dendritic cells to the tumor.²⁹ This also raises the point that some of the associated biomarkers identified for EL and other forms of CTCL may be expressed by infiltrating immune cells and not the tumor itself. Nevertheless, these seem to serve as sensitive and specific biomarkers for identifying early stage EL apart from other forms of interface dermatitis.

Other biologically relevant genes identified in our datasets correspond to both tumor and immune cell function. CD6, CD86, and CD8A were all upregulated, and are implicated in T-cell activation and regulation. MS4A1, a gene that encodes the B-cell marker CD20, was upregulated in EL. PTPRC was also upregulated, and is essential for T- and B-cell antigen receptor signaling. Immunoregulatory cytokines and their receptors were upregulated including IL6 and IL21R. S100A12 regulates inflammatory and immune responses, and was upregulated in EL. Tumor necrosis factor alpha (TNF-alpha) has been implicated in the pathogenesis of CTCL.³⁰ TNF, TNFRSF17, CD27, and FASLG were all significantly upregulated in canines with EL. CD27, a TNF family member required for the maintenance of T-cell immunity, was significantly upregulated. FoxP3 has been reported to be variably expressed depending on the stage of disease in CTCL.³¹ FoxP3 was not significantly differentiated in the EL canines compared to the healthy controls; however two canines, Sample 3 and Sample 4, had relatively greater counts than all other cases. They represent cases with significant dermal neoplastic involvement and patch/plaque stage, respectively. Last, given that CCL22 was upregulated, CCR4 depleting antibodies like mogamulizumab may be helpful for treating canine EL.

We also observed downregulated genes that may be biologically significant in EL. VGLL3 has been recognized as a tumor suppressor gene in serous ovarian carcinomas^{32,33} and stomach adenocarcinoma.³⁴ VGLL3, which regulates the Hippo pathway,³⁵ was downregulated in EL skin. IL16 was also downregulated, reflecting the pattern observed in severe Sezary Syndrome.³⁶ Reintroduction of pro-IL16 in MOLT4 tumors can induce regression in nude mice.³⁷ Therefore, adding back tumor suppressor genes may serve as a therapeutic option for EL and should be considered in future veterinary clinical trials.

Forms of CTCL that express CD8+ phenotype in humans have been reported³¹: in some rare cases, well-defined types of CTCL (such as MF) express CD8+ and have similar clinical presentation and disease prognosis as the more common CD4+ cases.³⁸ Outside of these, studies suggest separate groups of more aggressive CD8+ cytotoxic CTCLs,³⁹ including an epidermotropic type and a panniculitis-like subcutaneous T cell lymphoma type. Therefore, it is possible that, genetically and/or transcriptionally, canine EL matches human CD8+ lymphomas more closely than MF as a general subtype. Future comparative oncology studies should further characterize these rare CD8+ tumors to better understand which condition is most closely modeled in dogs.

### Subjects

The present methods can be used to diagnose and treat CTCL (such as MF) in mammals, e.g., in veterinary subjects such as dogs, cats, or horses, or in humans, e.g., humans of color, e.g., humans who have skin of Fitzpatrick phototype IV, V, or VI. Human subjects will generally have a lesion, e.g., a skin rash that includes patches of exfoliative erythroderma, ulceration, plaques, and nodules; veterinary subjects can have depigmentation, crusting, erythema, ulceration and/or alopecia. Disease progression usually moves from patch stage to plaque stage to tumor stage.

### Methods of Diagnosis or Monitoring

The present methods rely on detection of two, three, four, or more genes selected from cathepsin W (CTSW), KLRK1 (NKG2D), TRAT1, and XCL1/XCL2 as biological makers of CTCL. The methods can include obtaining a sample from a subject, and evaluating the presence and/or level of two, three, four, or more of CTSW, KLRK1, TRAT1 and/or XCL1/XCL2 in the sample, and comparing the presence and/or level with one or more references, e.g., a control reference that represents a normal level of CTSW, KLRK1, TRAT1 and/or XCL1/XCL2, e.g., a level in an unaffected subject, and/or a disease reference that represents a level of CTSW, KLRK1, TRAT1 and/or XCL1/XCL2 associated with CTCL, e.g., a level in a subject having CTCL.

As used herein the term “sample”, when referring to the material to be tested for the presence of a biological marker using the method of the invention, includes inter alia tissue, e.g., a skin biopsy sample from a lesion suspected of being CTCL. Various methods are well known within the art for the identification and/or isolation and/or purification of a biological marker from a sample. An “isolated” or “purified” biological marker is substantially free of cellular material or other contaminants from the cell or tissue source from which the biological marker is derived i.e. partially or completely altered or removed from the natural state through human intervention. For example, nucleic acids contained in the sample can be isolated according to standard methods, for example using lytic enzymes, chemical solutions, or isolated by nucleic acid-binding resins following the manufacturer's instructions.

The present methods can include determining levels of either protein or mRNA encoding one, two, three, four, or more CTSW, KLRK1, TRAT1 and/or XCL1/XCL2. In some embodiments, the methods can also include determining levels of protein or mRNA encoding the aforementioned genes. Exemplary sequences for human, canine, feline, and Equine CTSW and TRAT1 are available in NCBI GenBank, e.g., as shown in Table A.

TABLE A
Exemplary sequences for CTSW, TRAT1, and KLRK1 (NKG2D)

| mRNA | protein | gene name | species |
|---|---|---|---|
| NM_001335.4 | NP_001326.3 | cathepsin W | Homo Sapiens |
| | | preproprotein | |
| NM_016388.4 | NP_057472.2 | T-cell receptor- | Homo Sapiens |
| | | associated | |
| | | transmembrane | |
| | | adapter 1 isoform 1 | |
| NM_001317747.2 | NP_001304676.1 | T-cell receptor- | Homo Sapiens |
| | | associated | |
| | | transmembrane | |
| | | adapter 1 isoform 2 | |
| NM_007360.4 | NP_031386.2 | NKG2-D type II | Homo Sapiens |
| | | integral membrane | |
| | | protein | |
| NM_002995.3 | NP_002986.1 | X-C motif | Homo Sapiens |
| | | chemokine ligand 1/ | |
| | | lymphotactin | |
| | | precursor | |
| NM_003175.4 | NP_003166.1 | X-C motif | Homo Sapiens |
| | | chemokine ligand 2/ | |
| | | cytokine SCM-1 beta | |
| | | precursor | |
| XM_038424941.1 | XP_038280869.1 | cathepsin W | Canis lupus |
| | | | familiaris |
| XM_038444882.1 | XP_038300810.1 | T-cell receptor- | Canis lupus |
| | | associated | familiaris |
| | | transmembrane | |
| | | adapter 1 | |
| XM_038439018.1 | XP_038294946.1 | NKG2-D type II | Canis lupus |
| | | integral membrane | familiaris |
| | | protein isoform X1 | |
| XM_038439017.1 | XP_038294945.1 | NKG2-D type II | Canis lupus |
| | | integral membrane | familiaris |
| | | protein isoform X1 | |
| XM_038439019.1 | XP_038294947.1 | NKG2-D type II | Canis lupus |
| | | integral membrane | familiaris |
| | | protein isoform X2 | |
| XM_038671439.1 | XP_038527367.1 | lymphotactin isoform | Canis lupus |
| | | X1 | familiaris |
| XM_038671435.1 | XP_038527363.1 | lymphotactin isoform | Canis lupus |
| | | X2 | familiaris |
| XM_038671436.1 | XP_038527364.1 | lymphotactin isoform | Canis lupus |
| | | X3 | familiaris |
| XM_038671437.1 | XP_038527365.1 | lymphotactin isoform | Canis lupus |
| | | X4 | familiaris |
| XM_038671438.1 | XP_038527366.1 | lymphotactin isoform | Canis lupus |
| | | X5 | familiaris |
| NM_001122871.1 | NP_001116343.1 | cathepsin W | Felis catus |
| | | precursor | |
| XM_006936023.5 | XP_006936085.1 | T-cell receptor- | Felis catus |
| | | associated | |
| | | transmembrane | |
| | | adapter 1 isoform X1 | |
| XM_045036901.1 | XP_044892836.1 | T-cell receptor- | Felis catus |
| | | associated | |
| | | transmembrane | |
| | | adapter 1 isoform X2 | |
| XM_003988411.4 | XP_003988460.3 | NKG2-D type II | Felis catus |
| | | integral membrane | |
| | | protein isoform X1 | |
| XM_045061359.1 | XP_044917294.1 | NKG2-D type II | Felis catus |
| | | integral membrane | |
| | | protein isoform X2 | |
| XM_001494633.4 | XP_001494683.1 | cathepsin W | Equus caballus |
| XM_005602016.3 | XP_005602073.1 | T-cell receptor- | Equus caballus |
| | | associated | |
| | | transmembrane | |
| | | adapter 1 | |
| XM_014740713.2 | XP_014596199.2 | NKG2-D type II | Equus caballus |
| | | integral membrane | |
| | | protein | |
| XM_005610937.3 | XP_005610994.2 | NKG2-D type II | Equus caballus |
| | | integral membrane | |
| | | protein | |
| XM_001490960.4 | XP_001491010.1 | lymphotactin | Equus caballus |

Where the sequence given above is a precursor, proprotein, or preproprotein, the methods can include detecting the mature sequence (e.g., amino acids 128-376 of NP_001326.3, which represents the mature human CTSW protein).

Preferably the presence and/or level of CTSW, KLRK1, TRAT1 and/or XCL1/XCL2 nucleic acids are evaluated, e.g., using methods known in the art, e.g., using polymerase chain reaction (PCR), reverse transcriptase polymerase chain reaction (RT-PCR), quantitative or semi-quantitative real-time RT-PCR, digital PCR i.e. BEAMing ((Beads, Emulsion, Amplification, Magnetics) Diehl (2006) Nat Methods 3:551-559); RNAse protection assay; Northern blot; various types of nucleic acid sequencing (Sanger, pyrosequencing, NextGeneration Sequencing); fluorescent in-situ hybridization (FISH); or gene array/chips) (Lehninger Biochemistry (Worth Publishers, Inc., current addition; Sambrook et al., Molecular Cloning: A Laboratory Manual (3^{rd} Edition, 2001); Bernard (2002) Clin Chem 48(8): 1178-1185; Miranda (2010) Kidney International 78:191-199; Bianchi (2011) EMBO Mol Med 3:495-503; Taylor (2013) Front. Genet. 4:142; Yang (2014) PLOS One 9(11):e110641); Nordstrom (2000) Biotechnol. Appl. Biochem. 31(2):107-112; Ahmadian (2000) Anal Biochem 280:103-110. In some embodiments, high throughput methods, e.g., protein or gene chips as are known in the art (see, e.g., Ch. 12, Genomics, in Griffiths et al., Eds. Modern genetic Analysis, 1999, W. H. Freeman and Company; Ekins and Chu, Trends in Biotechnology, 1999, 17:217-218; MacBeath and Schreiber, Science 2000, 289(5485):1760-1763; Simpson, Proteins and Proteomics: A Laboratory Manual, Cold Spring Harbor Laboratory Press; 2002; Hardiman, Microarrays Methods and Applications: Nuts & Bolts, DNA Press, 2003), can be used to detect the presence and/or level of CTSW, KLRK1, TRAT1 and/or XCL1/XCL2. Measurement of the level of a biomarker can be direct or indirect. For example, the abundance levels of CTSW, KLRK1, TRAT1 and/or XCL1/XCL2 can be directly quantitated. Alternatively, the amount of a biomarker can be determined indirectly by measuring abundance levels of cDNA, amplified RNAs or DNAs, or by measuring quantities or activities of RNAs, or other molecules that are indicative of the expression level of the biomarker. In some embodiments a technique suitable for the detection of alterations in the structure or sequence of nucleic acids, such as the presence of deletions, amplifications, or substitutions, can be used for the detection of biomarkers of this invention.

RT-PCR can be used to determine the expression profiles of biomarkers (U.S. Patent No. 2005/0048542A1). The first step in expression profiling by RT-PCR is the reverse transcription of the RNA template into cDNA, followed by its exponential amplification in a PCR reaction (Ausubel et al (1997) Current Protocols of Molecular Biology, John Wiley and Sons). To minimize errors and the effects of sample-to-sample variation, RT-PCR is usually performed using an internal standard, which is expressed at constant level among tissues, and is unaffected by the experimental treatment. Housekeeping genes, such as GAPDH, B2M, cg14980/CCZ1, HPRT, and RPL13A, are commonly used.

Gene arrays can be prepared by selecting probes that comprise polynucleotide sequences that bind to CTSW, KLRK1, TRAT1, and/or XCL1/XCL2 and then immobilizing such probes to a solid support or surface. For example, the probes may comprise DNA sequences, RNA sequences, co-polymer sequences of DNA and RNA, DNA and/or RNA analogues, or combinations thereof. The probe sequences can be synthesized either enzymatically in vivo, enzymatically in vitro (e.g. by PCR), or non-enzymatically in vitro.

Alternatively, the presence and/or level of CTSW, KLRK1, TRAT1 and/or XCL1/XCL2 proteins can be evaluated using methods known in the art, e.g., using standard electrophoretic and quantitative immunoassay methods for proteins, including but not limited to, Western blot; enzyme linked immunosorbent assay (ELISA); biotin/avidin type assays; protein array detection; radio-immunoassay; immunohistochemistry (IHC); immune-precipitation assay; FACS (fluorescent activated cell sorting); mass spectrometry (Kim (2010) Am J Clin Pathol 134:157-162; Yasun (2012) Anal Chem 84(14):6008-6015; Brody (2010) Expert Rev Mol Diagn 10(8):1013-1022; Philips (2014) PLOS One 9(3):e90226; Pfaffe (2011) Clin Chem 57(5): 675-687). The methods typically include revealing labels such as fluorescent, chemiluminescent, radioactive, and enzymatic or dye molecules that provide a signal either directly or indirectly. As used herein, the term “label” refers to the coupling (i.e. physically linkage) of a detectable substance, such as a radioactive agent or fluorophore (e.g. phycoerythrin (PE) or indocyanine (Cy5), to an antibody or probe, as well as indirect labeling of the probe or antibody (e.g. horseradish peroxidase, HRP) by reactivity with a detectable substance.

In some embodiments, an ELISA method may be used, wherein the wells of a microtiter plate are coated with an antibody against which the protein is to be tested. The sample containing or suspected of containing the biological marker is then applied to the wells. After a sufficient amount of time, during which antibody-antigen complexes would have formed, the plate is washed to remove any unbound moieties, and a detectably labelled molecule is added. Again, after a sufficient period of incubation, the plate is washed to remove any excess, unbound molecules, and the presence of the labeled molecule is determined using methods known in the art. Variations of the ELISA method, such as the competitive ELISA or competition assay, and sandwich ELISA, may also be used, as these are well-known to those skilled in the art.

In some embodiments, an IHC method may be used. IHC provides a method of detecting a biological marker in situ. The presence and exact cellular location of the biological marker can be detected. Typically a sample is fixed with formalin or paraformaldehyde, embedded in paraffin, and cut into sections for staining and subsequent inspection by confocal microscopy. Current methods of IHC use either direct or indirect labelling. The sample may also be inspected by fluorescent microscopy when immunofluorescence (IF) is performed, as a variation to IHC.

Mass spectrometry, and particularly matrix-assisted laser desorption/ionization mass spectrometry (MALDI-MS) and surface-enhanced laser desorption/ionization mass spectrometry (SELDI-MS), is useful for the detection of biomarkers of this invention (See U.S. Pat. Nos. 5,118,937; 5,045,694; 5,719,060; 6,225,047).

In some embodiments, the level of CTSW, KLRK1, TRAT1 and/or XCL1/XCL2 is comparable to or above the level of the nucleic acids or protein(s) in the disease reference, then the subject can be diagnosed with CTCL.

Suitable reference values can be determined using methods known in the art, e.g., using standard clinical trial methodology and statistical analysis. The reference values can have any relevant form. In some cases, the reference comprises a predetermined value for a meaningful level of CTSW, KLRK1, TRAT1, and/or XCL1/XCL2 e.g., a control reference level that represents a normal level of CTSW, KLRK1, TRAT1, and/or XCL1/XCL2 e.g., a level in an unaffected subject or a subject who is not at risk of developing a disease described herein, or in a normal tissue (skin biopsy sample) of from the same subject, and/or a disease reference that represents a level of CTSW, KLRK1, TRAT1 and/or XCL1/XCL2 associated with CTCL.

The predetermined level can be a single cut-off (threshold) value, such as a median or mean, or a level that defines the boundaries of an upper or lower quartile, tertile, or other segment of a clinical trial population that is determined to be statistically different from the other segments. It can be a range of cut-off (or threshold) values, such as a confidence interval. It can be established based upon comparative groups, such as where association with risk of developing disease or presence of disease in one defined group is a fold higher, or lower, (e.g., approximately 2-fold, 4-fold, 8-fold, 16-fold or more) than the risk or presence of disease in another defined group. It can be a range, for example, where a population of subjects (e.g., control subjects) is divided equally (or unequally) into groups, such as a low-risk group, a medium-risk group and a high-risk group, or into quartiles, the lowest quartile being subjects with the lowest risk and the highest quartile being subjects with the highest risk, or into n-quantiles (i.e., n regularly spaced intervals) the lowest of the n-quantiles being subjects with the lowest risk and the highest of the n-quantiles being subjects with the highest risk.

In some embodiments, the predetermined level is a level or occurrence in the same subject, e.g., at a different time point, e.g., an earlier time point.

Subjects associated with predetermined values are typically referred to as reference subjects. For example, in some embodiments, a healthy control reference subject does not have a disorder described herein (e.g. CTCL).

A disease reference subject is one who has (or has an increased risk of developing) CTCL. An increased risk is defined as a risk above the risk of subjects in the general population.

Thus, in some cases the level of CTSW, KLRK1, TRAT1 and/or XCL1/XCL2 in a subject being greater than a healthy (disease-free) reference level of CTSW, KLRK1, TRAT1 and/or XCL1/XCL2 is indicative of a clinical status (e.g., indicative of the presence of CTCL). In other cases the level of CTSW, KLRK1, TRAT1 and/or XCL1/XCL2 in a subject being less than or equal to the healthy reference level of CTSW, KLRK1, TRAT1 and/or XCL1/XCL2 is indicative of the absence of disease or normal risk of the disease. In some embodiments, the amount by which the level in the subject is the less than the reference level is sufficient to distinguish a subject from a control subject, and optionally is a statistically significantly less than the level in a control subject. In cases where the level of CTSW, KLRK1, TRAT1 and/or XCL1/XCL2 in a subject being equal to the reference level of CTSW, KLRK1, TRAT1, and/or XCL1/XCL2 the “being equal” refers to being approximately equal (e.g., not statistically different).

The predetermined value can depend upon the particular population of subjects (e.g., human subjects) selected. Accordingly, the predetermined values selected may take into account the category (e.g., sex, age, health, risk, presence of other diseases) in which a subject (e.g., human subject) falls. Appropriate ranges and categories can be selected with no more than routine experimentation by those of ordinary skill in the art.

In characterizing likelihood, or risk, numerous predetermined values can be established.

In some embodiments, the methods include monitoring a treatment for efficacy, e.g., by determining a first level of CTSW, KLRK1, TRAT1 and/or XCL1/XCL2; administering a treatment for CTCL, e.g., as described herein, and then determining a second level of CTSW, KLRK1, TRAT1 and/or XCL1/XCL2. If the second level of CTSW, KLRK1, TRAT1 and/or XCL1/XCL2 is below the first level, the treatment has been successful. In some embodiments, if the second level of level of CTSW, KLRK1, TRAT1 and/or XCL1/XCL2 is less than or equal to a healthy control level, the healthcare provide can consider discontinuing the treatment. The methods can also include identifying the subject as having CTCL based on the first level of CTSW, KLRK1, TRAT1 and/or XCL1/XCL2 being above a healthy control reference level.

### Kits

Also provided herein are kits for use in a method described herein. Such kits can includes reagents for detecting the biomarkers described herein, e.g., probes or primers that bind to two, three, or all four of CTSW, KLRK1, TRAT1 and/or XCL1/XCL2, and optionally one or more housekeeping genes for use as controls, e.g., B2M, RPL13A, CCZ1, HPRT1, and/or GAPDH. In some embodiments, the kits don't include probes or primers that bind to any other biomarkers. In some embodiments, the kits comprise a microarray. In some embodiments, the probes or primers bind to at least 20 consecutive nucleotides of a sequence shown in Table 4, or a human homolog thereof. In some embodiments, the probes or primers are detectably labeled (e.g., with a fluorophore or other detectable moiety) or comprise a barcode or other unique molecular identifier that allows the presence of the target sequence to be identified.

### Methods of Treatment

Once a subject has been identified as having CTCL using a method described herein, a treatment can be selected and/or the subject can be treated accordingly. Therapy options can comprise skin-directed therapies, such as topical steroids, involved-site radiation therapy (RT), total skin electron-beam therapy (TSEBT), or phototherapy/photopheresis, and systemic therapies, such as immunotherapies (e.g., retinoids, interferon, chimeric antigen receptor T cells), small molecules (histone deacetylase inhibitors), antibodies (e.g., anti-CCR4 (e.g., mogamulizumab), anti-programmed cell death protein 1, anti-CD47, or anti-CD30 (e.g., brentuximab vedotin)), or chemotherapy. Allogeneic haematopoietic stem cell transplantation can also be used, see, e.g., Dummer et al., Nat Rev Dis Primers. 2021 Aug. 26; 7(1):61; Kempf and Mitteldorf, Hematol Oncol. 2021 June; 39 Suppl 1:46-51; Weiner et al., J Am Acad Dermatol. 2021 March; 84(3):597-604; and Mehta-Shah, J Natl Compr Canc Netw. 2020 May; 18(5):522-536.

### EXAMPLES

The invention is further described in the following examples, which do not limit the scope of the invention described in the claims.

### Materials & Methods

The following materials and methods were used in the Examples herein.

Clinical samples: Skin biopsies from the biorepository at Tufts Cummings School were selected based on pathology reports. H&E sections were reexamined by a board-certified veterinary pathologist to confirm diagnoses and absence of obvious infectious disease, and clinical notes were reexamined by a board-certified veterinary dermatologist. Healthy control samples were obtained from leg margin biopsies from amputations. For the discovery cohort, six epitheliotropic lymphoma samples were obtained from shave and/or punch biopsies of dogs as noted in the case presentation section. Samples were deposited with written owner consent in the Tufts biobank at the time the veterinary patients were seen at the hospital, spanning the years 2011-2019. For the validation cohort, eight EL samples were obtained from the biobank at Colorado State University, seven of which yielded enough RNA for downstream analyses.

Isolation of RNA from FFPE blocks: 30 μm curls were cut from the blocks and stored in Eppendorf tubes at ambient temperature. RNA was isolated using the Qiagen FFPE RNeasy kit per the manufacturer directions. Briefly, razor blades were treated with RNase, excess paraffin was removed, and tissues were sliced into thin strips (5 μm) to create more surface area prior to incubation with deparaffinization solution (Qiagen). The manufacturer protocol was followed and RNA was quantified using a nanodrop.

Nanostring cartridge and processing: A custom Nanostring canine gene panel of 160 genes including cytokine, chemokine, and immune genes, as well as skin and immune cell specific transcripts was created. We used B2M, RPL13A, CCZ1 and HPRT as housekeeping genes for this study. For the validation cohort, the NanoString canine Immune-Oncology (IO) panel was used. RNA was hybridized using a BioRad C1000 touch machine, and samples were loaded into Nanostring cartridges and analyzed with a Sprint machine. Gene expression data are deposited on GEO under Accession #GSE213087.

nSolver analysis: NanoString's software, nSolver was used for all NanoString analysis. Raw counts were plotted with GraphPad Prism. Advanced analysis was used for the “cell Type Score”, which is a summary statistic of the expression of the marker genes for each cell type. It is the geometric mean of the log 2-transformed normalized counts for each set of marker genes. These scores were validated against FACS and IHC, and are a robust method of quantifying relative cell type abundance.⁶

Immunohistochemistry (IHC): IHC was performed on 5 μm sections using rabbit-anti-canine CD244, CTSW (US Biological) or isotype control (Biolegend) at 1:100 dilution using a Dako automated slide staining machine. All sections were counterstained with hematoxylin. H&E images were taken using an Olympus BX51 microscope with Nikon NIS Elements software version 3.10, and IHC images were taken using an Olympus BX40 microscope with cellSens Entry software version 1.14.

qPCR: qPCR for CTSW was performed on remaining RNA samples or frozen lymphoma samples using BioRad iScript cDNA synthesis kit and iTaq universal SYBR green supermix per the manufacturer's protocols. A 60 degree qPCR program was run on QuantStudio (Applied Biosystems) or BioRad CFX96 machine.

Comparison to human CTCL: The human CTCL dataset from Nielsen et al.⁷ (GSE143382) was analyzed using Geo2R. Gene lists were truncated in Microsoft Excel using the formula “=IF(ISERROR(VLOOKUP(cell,reference,1,FALSE)), FALSE,TRUE)” and shared DEGs between human and canine were analyzed with BioVenn.

Statistics: To assess the statistical significance of our results, we performed DEG analysis using Rosalind software and/or nSolver software. We also analyzed raw and/or normalized counts between groups using nSolver and GraphPad Prism software version 9 to examine potential differences in previously identified genes pertinent to EL and CTCL pathogenesis. Normality tests were performed in GraphPad Prism. Normally distributed data were analyzed using student's t test and non-normally distributed data were analyzed using Mann-Whitney U test. Receiver Operator Characteristic (ROC) curves were calculated in GraphPad Prism. Multi-ROC curves were calculated by a biostatistician in SPSS. A statistically significant difference was considered as p<0.05.

### Example 1. Cathepsin W, T-Cell Receptor-Associated Transmembrane Adapter 1 (TRAT1) and Killer Cell Lectin Like Receptor K1 (KLRK1) are Sensitive and Specific RNA Biomarkers of Canine Epitheliotropic Lymphoma

RNA from FFPE skin curls were isolated from six canines with EL, and five healthy canine patients. Using the NanoString nCounter software, we performed comparative transcriptomics studies on 160 genes from lesional skin biopsies from 6 canine EL cases and 5 healthy canine cases. Our NanoString probeset targets included cytokine, chemokine, and immune related genes, as well as skin associated genes and neuroendocrine genes. These 160 genes were examined based on their known cellular functions. We then compared our EL findings to a previously published CTCL dataset (Human Dataset GSE143382). Principal component analysis (PCA) revealed methods to predict disease status.

### Example 1.1. Canine Gene Expression Analysis Reveals Significant Increases and Decreases in Key Immune and Skin Genes in EL Versus Healthy Controls

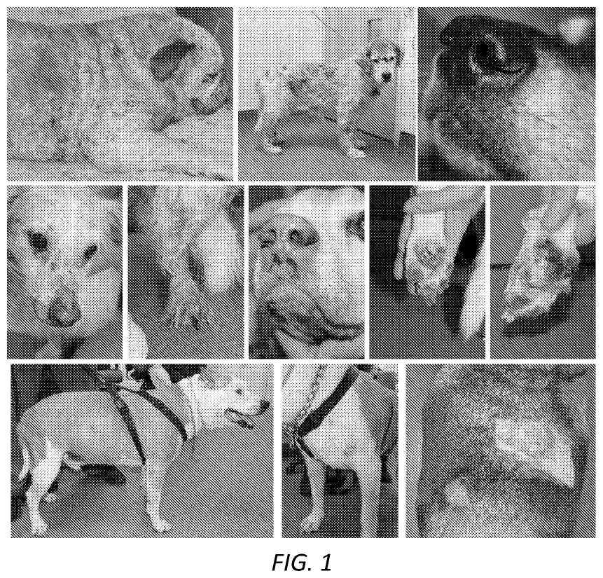

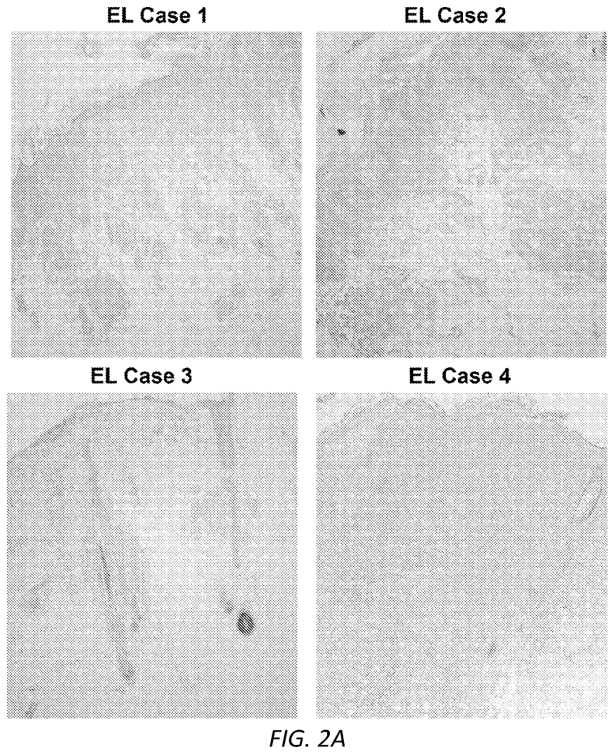

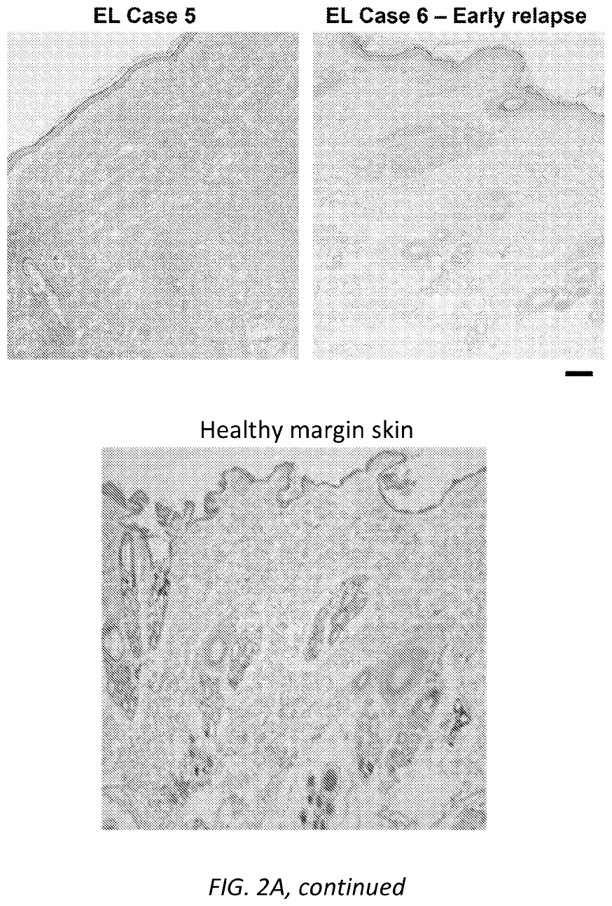

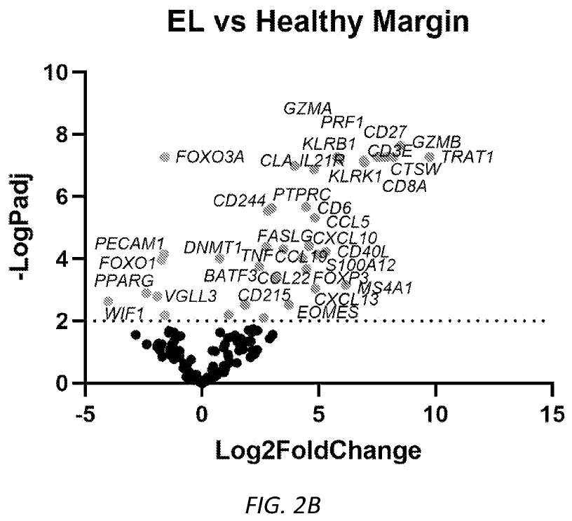

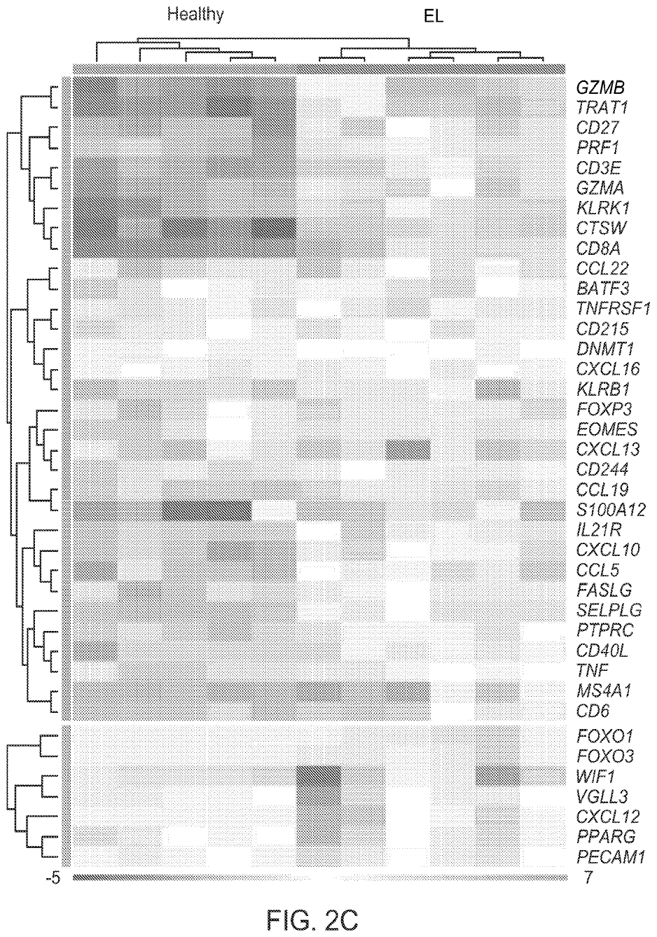

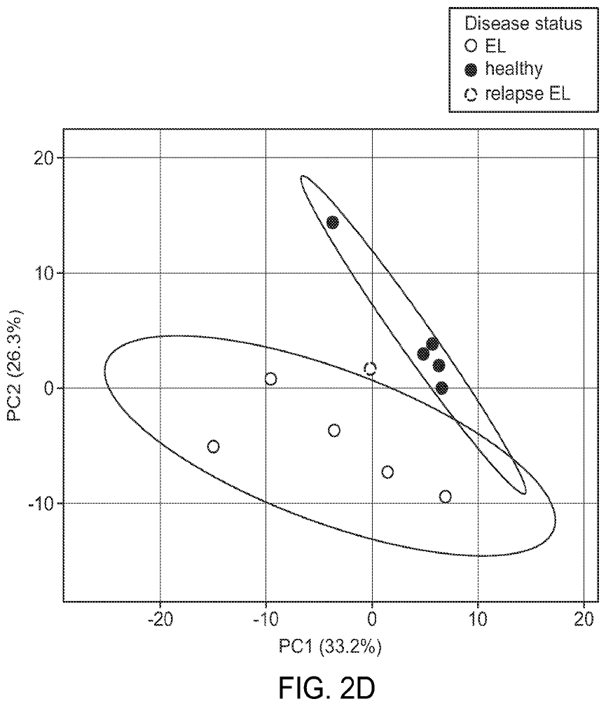

Canine EL can present with different features including depigmentation, crusting, erythema, ulceration and/or alopecia (FIG. 1). We analyzed tissue blocks leftover from diagnostic biopsies to assess gene expression (FIG. 2A, Table 1). Using a NanoString platform that was optimized for FFPE RNA analysis, we performed targeted transcriptomics studies on 160 custom curated genes from lesional skin biopsies from 6 canine EL cases and 5 healthy canine cases. Our NanoString probeset targets included cytokine, chemokine, and immune related genes, as well as skin associated genes and neuroendocrine genes. Advanced cell type analysis revealed cytotoxic T cells were significantly abundant (p=0.0001; not shown). Comparing the EL samples to healthy margins revealed 32 upregulated differentially expressed genes (DEGs) and 7 down regulated DEGs with Padj<0.01 (FIGS. 2B & C). An array of genes involved in cytotoxic processes were all upregulated, including GZMA, KLRB1, KLRD1, KLRK1, and PRF1. CPA3, which is involved in proteolysis and degradation of endogenous proteins, was upregulated in EL lesions. Principal component analysis revealed that EL cases could be readily distinguished from healthy controls in 95% confidence intervals, with the exception of one early relapse case which fell in between the EL and healthy skin gene signatures (FIG. 2D).

TABLE 1
Discovery cohort signalments.

| Case & | | | Notes (relevant features in addition to |
|---|---|---|---|
| Diagnosis | Signalment* | Breed | neoplastic process) |
| EL Case 1 | 11 yo, | Dachshund | Patch/plaque stage |
| | MN | | |
| EL Case 2 | 13 yo, | Labrador | Multifocal ulceration with moderate |
| | MN | cross | neutrophilic inflammation |
| EL Case 3 | 11 yo, | Labrador | Significant eosinophilic infiltrate |
| | FS | Retriever | accompanying the neoplastic population; |
| | | | multifocal ulceration, serocellular crusts and |
| | | | intracorneal neutrophilic pustules |
| EL Case 4 | 9 yo, FS | Bloodhound | Significant dermal neoplastic involvement |
| EL Case 5 | 11 yo, | Olde English | Significant dermal neoplastic involvement |
| | FS | Bulldogge | with epitheliotropism restricted to the |
| | | | adnexa with relative sparing of the epidermis |
| EL Case 6 | 13 yo, | Golden | Early stage epitheliotropic lymphoma with |
| | MN | Retriever | minimal to absent dermal involvement. This |
| | | | is a recurrence from epitheliotropic |
| | | | lymphoma that had undergone remission |
| Healthy 1 | 8 yo, FS | Labrador | NA |
| | | Retriever | |
| Healthy 2 | 11 yo, | Siberian | NA |
| | FS | Husky cross | |
| Healthy 3 | 11 yo, | Golden | NA |
| | MN | Retriever | |
| Healthy 4 | 12 yo, | German | NA |
| | MN | Shepherd | |
| | | cross | |
| Healthy 5 | 6 yo, FS | Alaskan | NA |
| | | Malamute | |

*age at time of biopsy;
FS = female spayed, MN = male neutered

### Example 1.2. CTSW can Distinguish EL from Other Interface Dermatitis Conditions

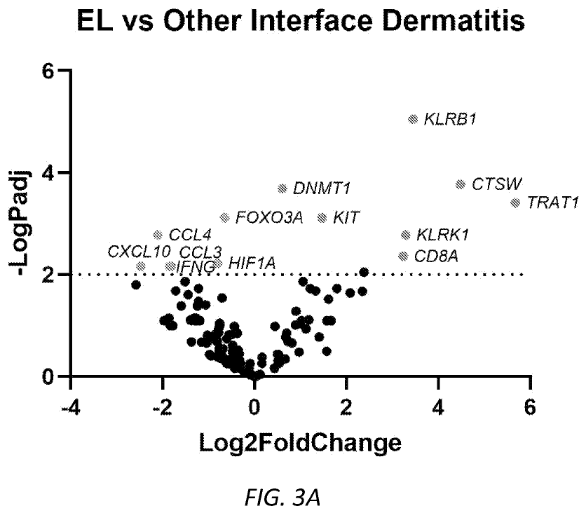

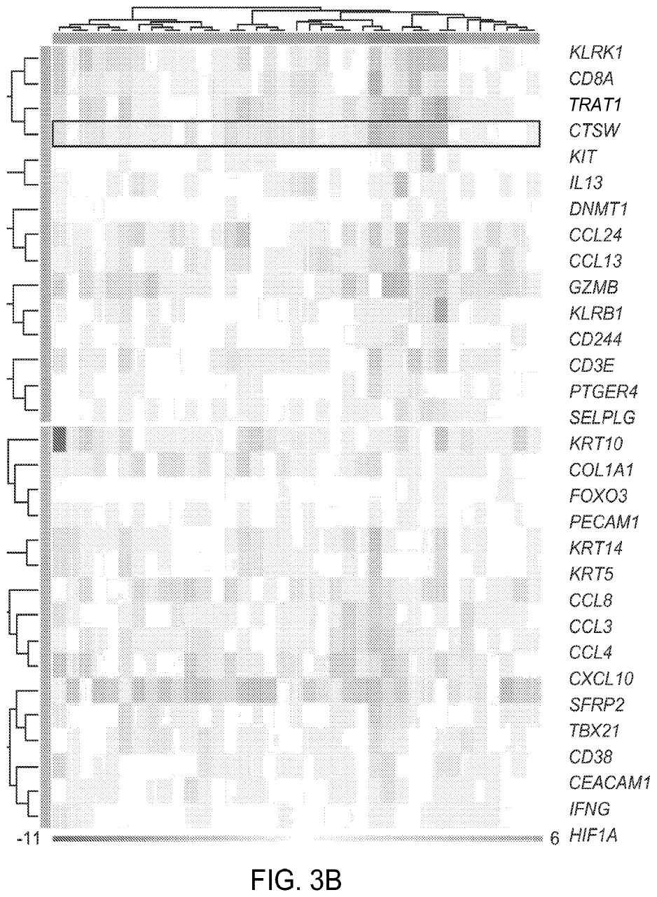

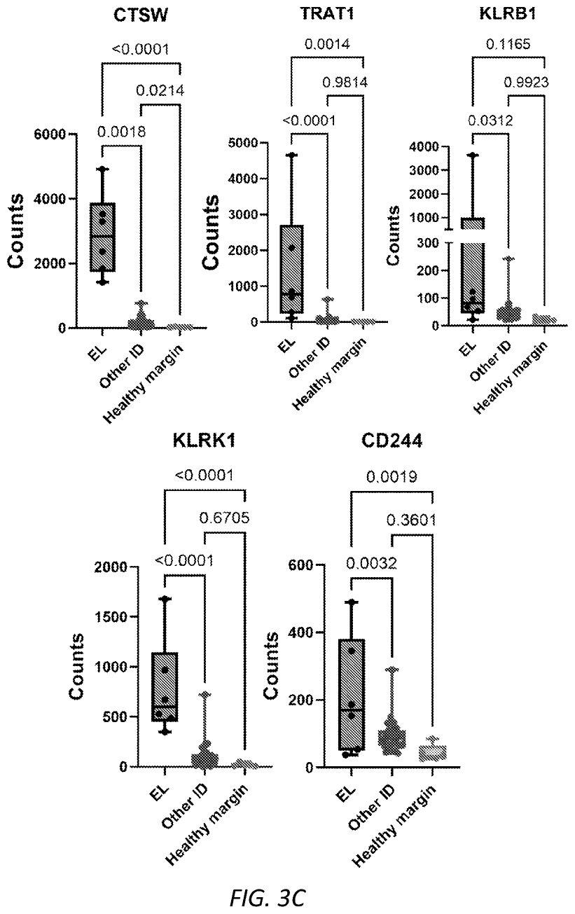

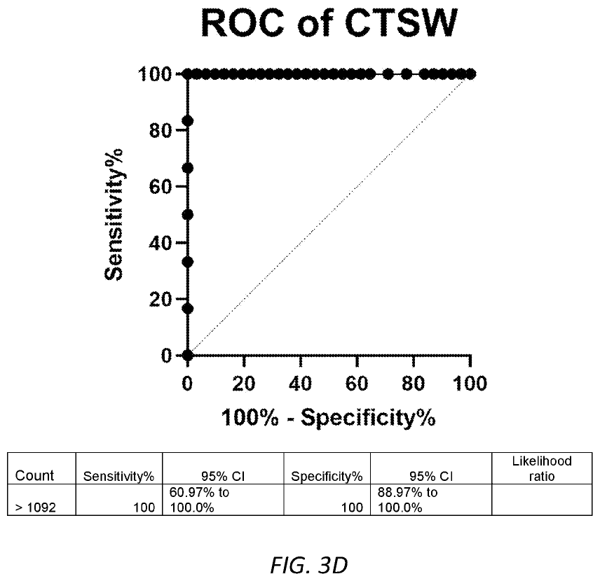

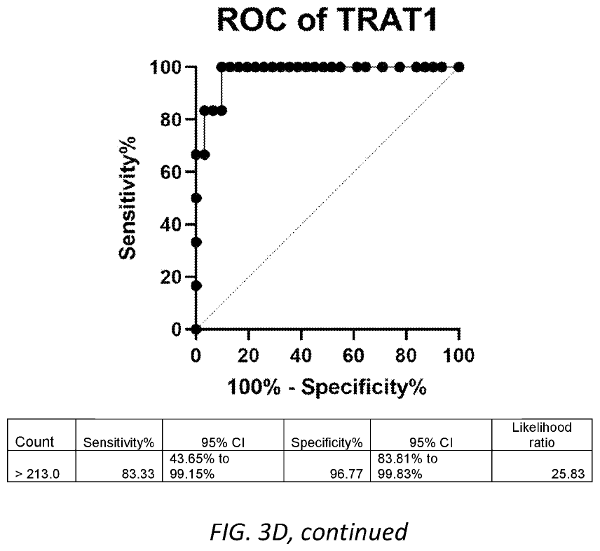

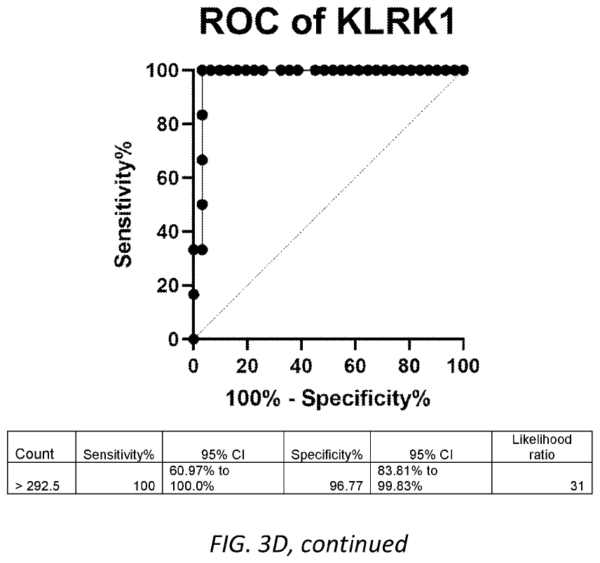

Next, we compared EL gene expression to other forms of interface dermatitis (ID) including lupus erythematosus, pemphigus subtypes, and erythema multiforme spectrum conditions. Seventeen DEGs were significantly downregulated and 14 were significantly upregulated with Padj<0.05 (FIGS. 3A & B). To evaluate whether any of these DEGs could serve as potential diagnostic biomarkers for EL, we analyzed RNA counts of the highest DEGs singly (FIG. 3C). Of these, CTSW exhibited no overlap between cases and cleanly distinguished EL from other potential clinical mimickers. Receiver operator characteristic (ROC) curves of CTSW was 100% sensitive and specific for counts >1092 (FIG. 3D). TRAT1 and KLRK1 were also highly significant (p<0.0001), and ROC analysis revealed 83.3% sensitivity and 96.77% specificity for TRAT1 counts >213, and 100% sensitivity and 96.77% specificity for KLRK1 counts >292.5.

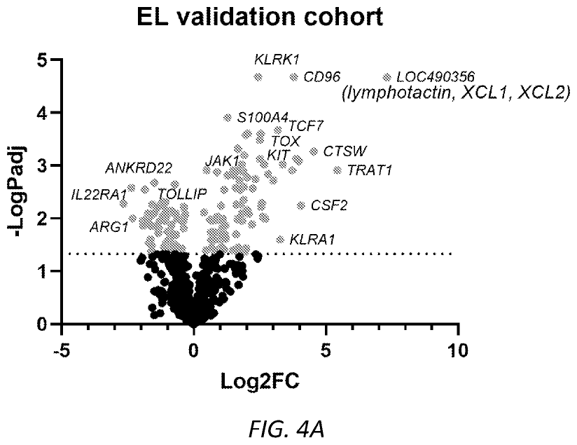

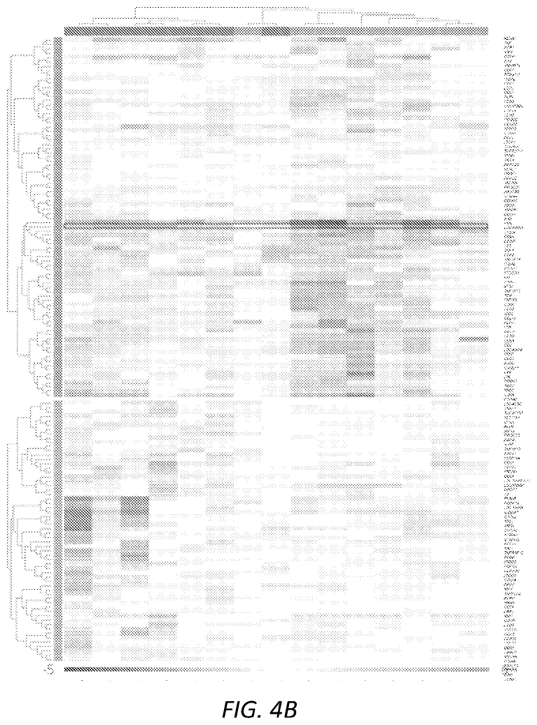

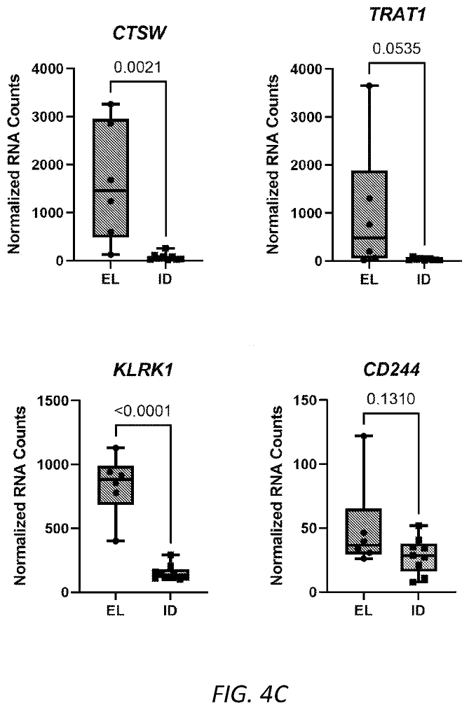

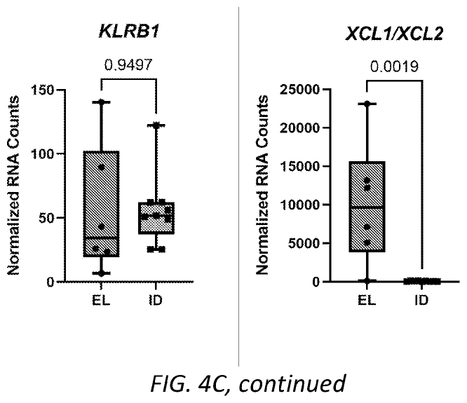

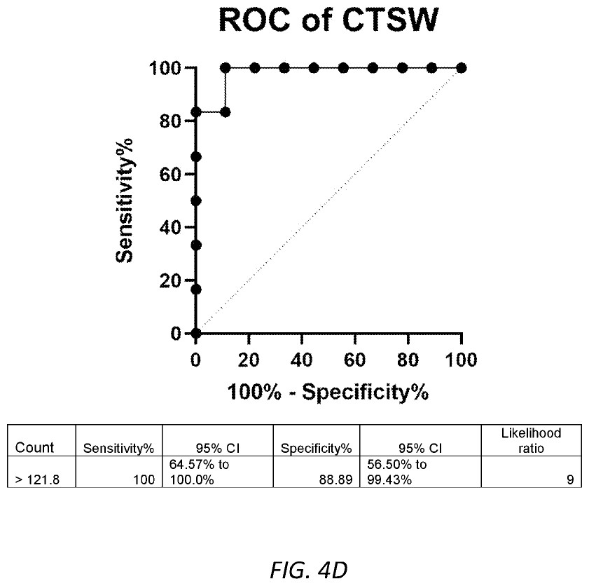

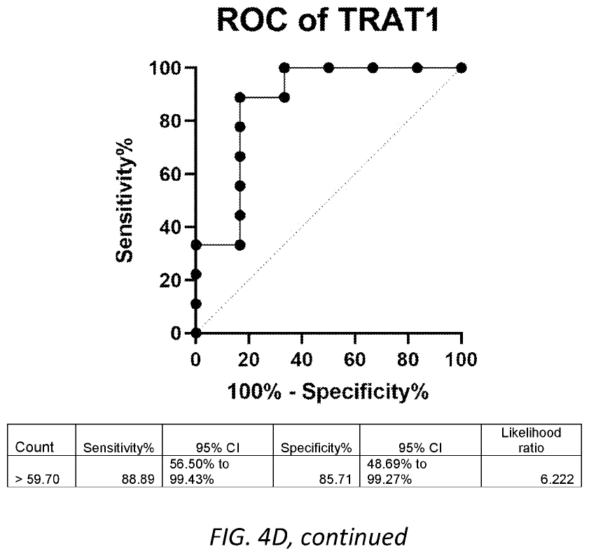

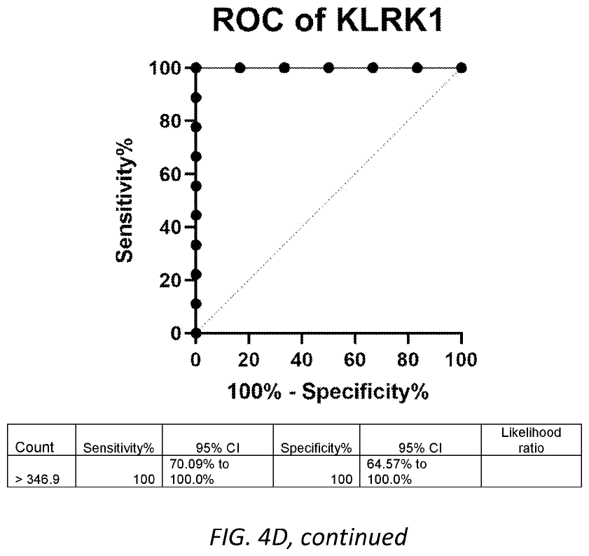

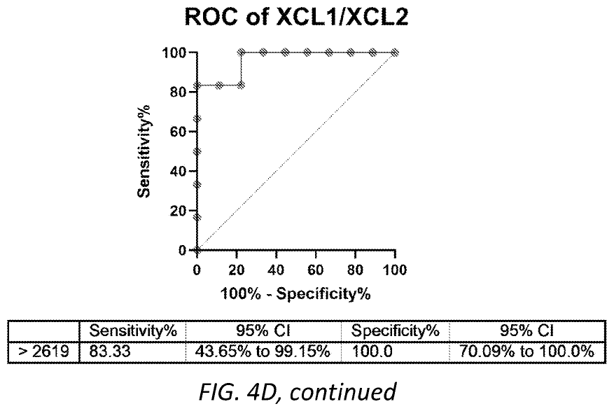

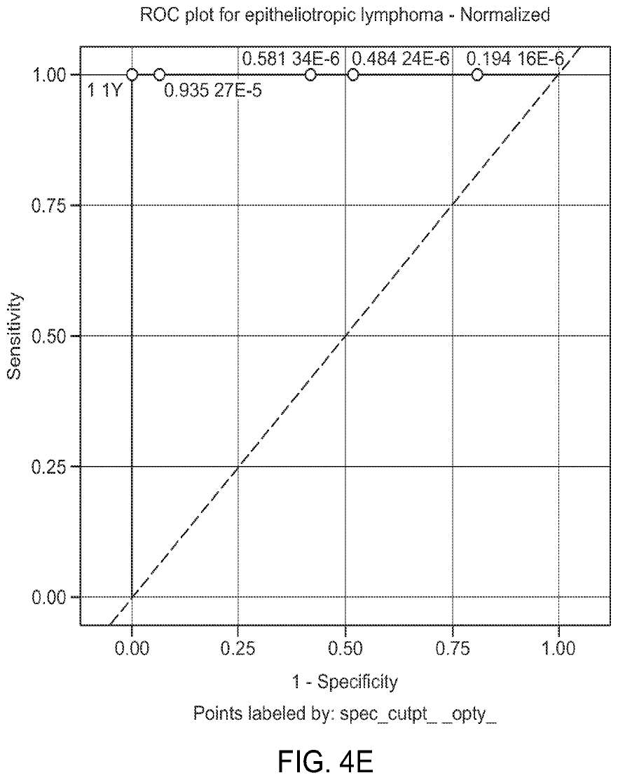

To confirm this finding, we performed analysis on a validation cohort of 6 EL and 9 ID samples using the NanoString canine IO panel (FIGS. 4A & B; Table 2). CTSW exhibited 100% sensitivity and 88.9% specificity to distinguish the two conditions at a count >121.8 (FIGS. 4C & D). TRAT1 exhibited 88.89% sensitivity and 83.33% specificity to distinguish EL from ID at a count >59.7. KLRK1 was 100% sensitive and specific at counts >346.9. The difference in absolute counts between the discovery cohort and the validation cohort is likely due to the 2 different platforms used (160 custom gene codeset versus canine IO ~800 gene codeset). We noted that an additional biomarker was identified by the canine IO panel: lymphotactin also called XCL1/XCL2, though we were unable to verify this gene in our discovery cohort because it was not included in the 160 gene codeset. Last, we examined whether combining biomarkers could be more powerful than using them singly. Multi-biomarker ROC analysis revealed that combining CTSW, TRAT1 and KLRK1 is highly sensitive and specific for identifying EL as compared to other forms of ID (FIG. 4E).

TABLE 2
Validation cohort signalments.

| Case & Diagnosis | Signalment* | Breed |
|---|---|---|
| EL Validation Case 1 | 13 yo spayed Female | Golden Retriever |
| EL Validation Case 2 | 17 yo spayed Female | Yorkshire Terrier |
| EL Validation Case 3 | 10 yo spayed Female | Shih Tzu |
| EL Validation Case 4 | 10 yo spayed Female | Labrador |
| | | Australian Shepherd |
| EL Validation Case 5 | 8 yo spayed Female | Cross |
| EL Validation Case 6 | 13.6 yo neutered Male | Golden Retriever |

*age at time of biopsy;
yo, year old

### Example 1.3. Comparative Analysis of Canine EL and Human CTCL Reveals Shared Inflammatory and Immunoregulatory Gene Expression Signatures

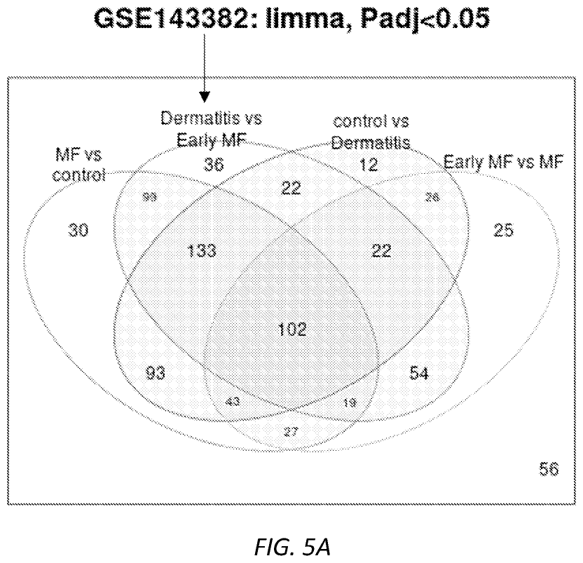

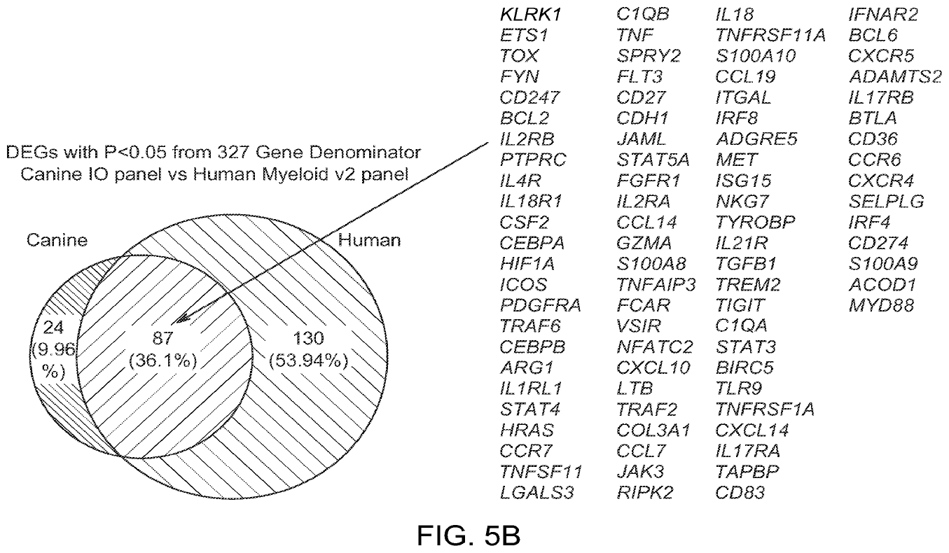

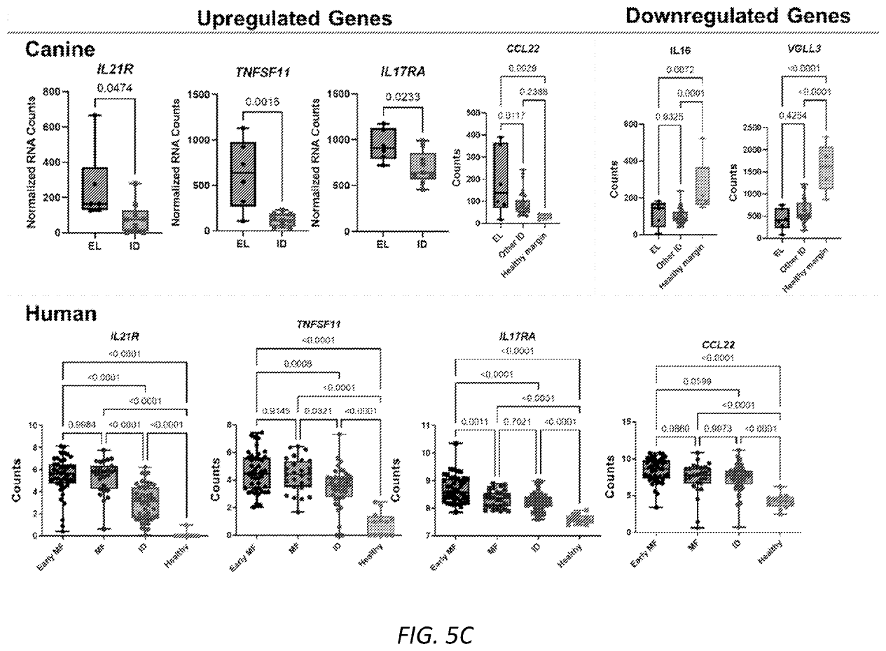

We also compared our EL findings to a previously published human CTCL dataset (Human Dataset GSE1433827). We focused on the DEGs between early MF and ID (FIG. 5A). First, we truncated the datasets to a common denominator gene list of 327 based on the NanoString panels (canine IO and human Myeloid v2). Next, we compared which of these genes had a P<0.05 and found 87 overlapping DEGs (FIG. 5B). We examined specific genes identified in our DEG overlap as well as other published genes of interest and found similar expression trends in human and canine datasets (FIG. 5C). We examined whether the two-gene classifier identified in human MF would also distinguish canine EL from ID, and found that while TOX and TRAF1 could separate the cases (green) from controls (black), better separation is achieved with CTSW and TRAT1 (FIG. 5D).

### Example 1.4. CTSW and CD244 are Expressed at the Protein Level in Canine EL and ID Lesions

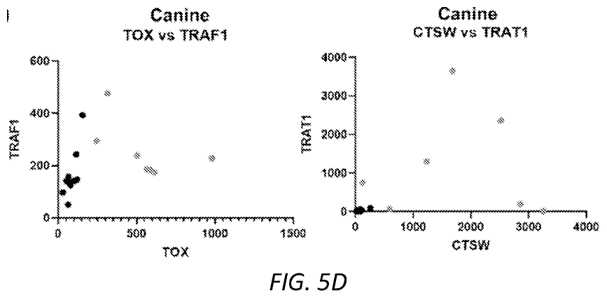

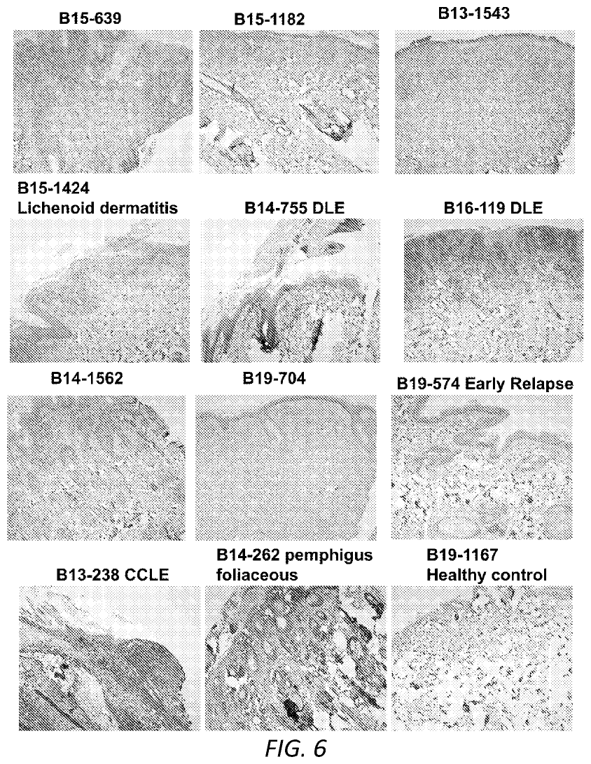

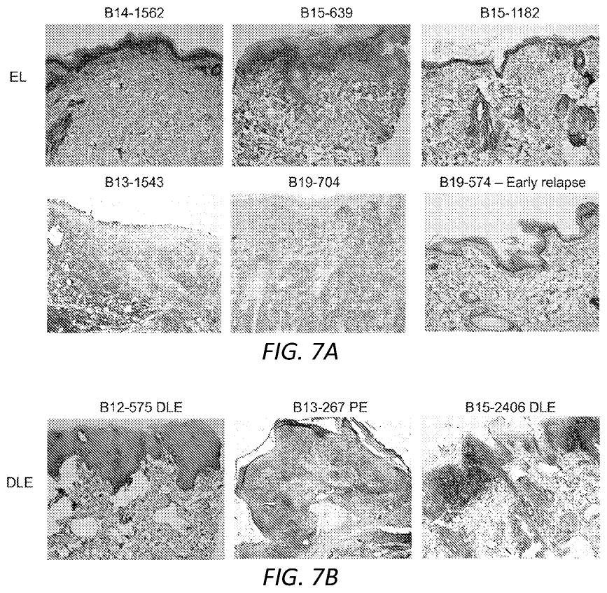

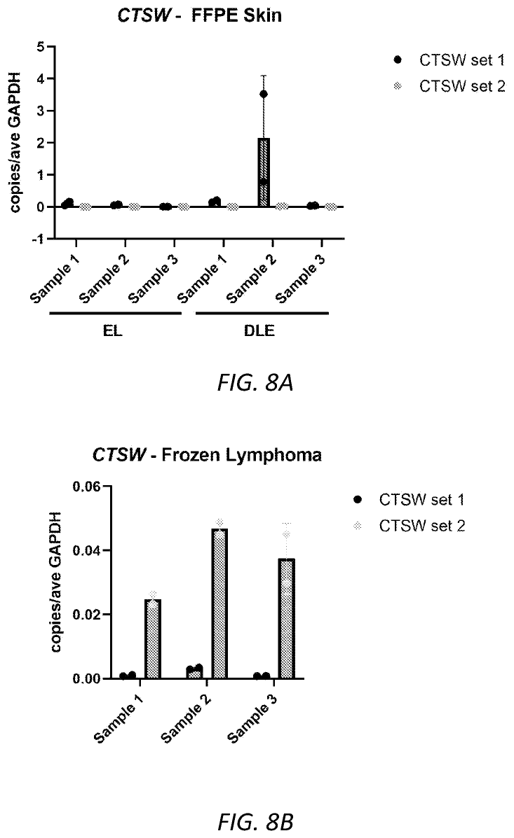

To confirm protein level expression of key genes, we performed IHC for CTSW and CD244 using antibodies that react to human and murine proteins with predicted homology to canine amino acid sequences, as no canine specific antibodies are commercially available. IHC staining did not differentiate EL from ID lesions, indicating possible cross-reactivity of the antibodies to other epitopes and/or discordance between RNA transcripts and protein level expression in the different conditions (FIGS. 6 & 7A-B). Similarly, qPCR for CTSW was not effective at distinguishing EL from ID in FFPE samples, which may be due to the highly fragmented RNA, but was informative in frozen samples. (FIGS. 8A-B; primers Table 3).

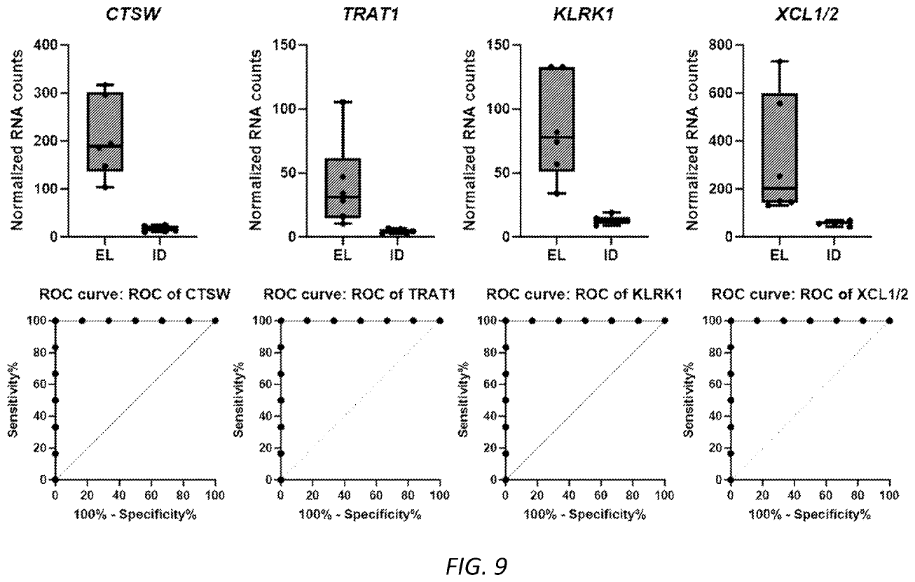

Thus, we developed a truncated custom nanostring nCounter microarray diagnostic tool with our 4 biomarkers (canine CTSW, TRAT1, KLRK1, and Loc490356/lymphotactin/XCL1/XCL2) and five housekeeping gene controls (canine B2M, RPL13A, CCZ1, HPRT1, and GAPDH) (target sequences shown in Table 4). This tool was able to sensitively and specifically detect EL in six dogs apart from six ID cases in FFPE samples (RNA was pulled from both discovery and validation cohort samples) (FIG. 9).

TABLE 3
qPCR Primers used for these studies.

| Name | Sequence | SEQ ID NO: |
|---|---|---|
| CTSW set 1 F | GCT TCC TGC TAC CCA CAC TT | 1 |
| CTSW set 1 R | AAG ATG TCC AGA CGA CGA GC | 2 |
| CTSW set 2 F | ACC CAT TCT TGG GGA ACA CC | 3 |
| CTSW set 2 R | AAC CCA CAA GCA GGA CAG AG | 4 |
| GAPDH F | GAT GGG CGT GAA CCA TGA G | 5 |
| GAPDH R | TCA TGA GGC CCT CCA CGA T | 6 |

| | NCBI | | |
|---|---|---|---|
| | Accession | | SEQ ID |
| Gene | No. | Target Sequence (Canis familiaris) | NO. |
| CTSW | XM_540846.5 | CCAGCCACTGGAGCTGAAACAGGTCTTT | 7 |
| | | GCGTTGTTCCAGATCCAGTACAACCGGA | |
| | | GTTACTCAAACCCAGAAGAATATGCTCG | |
| | | TCGTCTGGACATCTTT | |
| TRAT1 | XM_535733.5 | GTACAGTTACTCTGAAGACTATATTCCCA | 8 |
| | | GGGAGGAGGAGTATTATGTTGAAGACAC | |
| | | ACCAATTTATGGTAACTTAGATGACATGG | |
| | | TCCCAGAACCAGTG | |
| KLRK1 | XM_005637160.1 | CCTTAATTATAACTCTGAACCTGTAACAC | 9 |
| | | ATGGTACTTCTCCACGAAGGCAAAAGAG | |
| | | AAAGCCTACATTAATCACCAGCAAATGTG | |
| | | GAGAAAACTCATCT | |
| Loc490356/ | XM_022421696.1 | TTTCTCACTGAATTCATCGTGGAAGGTGT | 10 |
| lymphotactin/ | | GGGGAGTGAAGTCCTAGAAAGGAGCAT | |
| XCL1/XCL2 | | CTGTATGAGTCTGACGACCAAGAGACTG | |
| | | CCAGTTAAAAGCATCA | |
| B2M | NM_001284479.1 | TCTTCTGGTCCACACTGAATTCACTCCCA | 11 |
| | | ATGAGCAGGATGAGTTTAGCTGCCGTGT | |
| | | AAAGCATGTTACTCTCAGTGAGCCCCAG | |
| | | ATCGTTAAGTGGGAC | |
| RPL13A | NM_001313766.1 | GGCCGGAAGGTTGTAGTCGTGCGCTGT | 12 |
| | | GAAGGCATCAACATTTCTGGCAATTTCTA | |
| | | CAGAAACAAGTTGAAGTACCTGGCCTTC | |
| | | CTCCGCAAGCGGATGA | |
| CCZ1 | XM_536878.6 | GCCGAGAAAAGTACAATTCACATGAGGA | 13 |
| | | AAACACCTAGTGTGTCACTTACATCTGTG | |
| | | CATCCGGACTTAATGAAGATTCTGGGTG | |
| | | ACATCAACAGTGATT | |
| HPRT1 | NM_001003357.1 | CCATCACATCGTAGCCCTCTGTGTGCTC | 14 |
| | | AAGGGAGGCTATAAATTCTTTGCTGACC | |
| | | TGCTGGATTATATCAAAGCACTGAACAG | |
| | | AAATAGTGATGGATCC | |
| GAPDH | NM_001003142.1 | CAGTGACACCCACTCTTCCACCTTCGAC | 15 |
| | | GCCGGGGCTGGCATTGCCCTCAATGAC | |
| | | CACTTTGTCAAGCTCATTTCCTGGTATGA | |
| | | CAATGAATTTGGCTAC | |

### REFERENCES

1. Willemze R, Cerroni L, Kempf W, Berti E, Facchetti F, Swerdlow S H, et al. The 2018 update of the WHO-EORTC classification for primary cutaneous lymphomas. Blood. 2019; 133:1703-14.
2. Muñoz-González H, Molina-Ruiz A M, Requena L. Clinicopathologic Variants of Mycosis Fungoides. Actas Dermosifiliogr. 2017; 108:192-208.
3. Criscione V D, Weinstock M A. Incidence of Cutaneous T-Cell Lymphoma in the United States, 1973-2002. Arch Dermatol. 2007; 143:854-9.
4. Fontaine J, Bovens C, Bettenay S, Mueller R S. Canine cutaneous epitheliotropic T-cell lymphoma: a review. Vet Comp Oncol. 2009; 7:1-14.
5. Moore P F, Olivry T, Naydan D. Canine cutaneous epitheliotropic lymphoma (mycosis fungoides) is a proliferative disorder of CD8+ T cells. Am J Pathol. 1994; 144:421-9.
6. Danaher P, Warren S, Dennis L, D'Amico L, White A, Disis M L, et al. Gene expression markers of Tumor Infiltrating Leukocytes. J Immunother Cancer. 2017:5:18.
7. Nielsen P R, Eriksen J O, Lindahl L M, Wehkamp U, Bzorek M, Andersen G, et al. Diagnostic Two-Gene Classifier in Early-Stage Mycosis Fungoides: A Retrospective Multicenter Study. J Invest Dermatol. 2021; 141:213-7.e5.
8. Olson O C, Joyce J A. Cysteine cathepsin proteases: regulators of cancer progression and therapeutic response. Nat Rev Cancer. 2015; 15:712-29.
9. Dheilly E, Battistello E, Katanayeva N, Sungalee S, Michaux J, Duns G, et al. Cathepsin S Regulates Antigen Processing and T Cell Activity in Non-Hodgkin Lymphoma. Cancer Cell. 2020; 37:674-89.e12.
10. Bararia D, Hildebrand J A, Stolz S, Haebe S, Alig S, Trevisani C P, et al. Cathepsin S Alterations Induce a Tumor-Promoting Immune Microenvironment in Follicular Lymphoma. Cell Rep. 2020; 31:107522.
11. Linnevers C, Smeekens S P, Brömme D. Human cathepsin W, a putative cysteine protease predominantly expressed in CD8+ T-lymphocytes. FEBS Lett. 1997; 405:253-9.
12. Wex T, Bühling F, Wex H, Günther D, Malfertheiner P, Weber E, et al. Human cathepsin W, a cysteine protease predominantly expressed in NK cells, is mainly localized in the endoplasmic reticulum. J Immunol. 2001; 167:2172-8.
13. Stoeckle C, Gouttefangeas C, Hammer M, Weber E, Melms A, Tolosa E. Cathepsin W expressed exclusively in CD8+ T cells and NK cells, is secreted during target cell killing but is not essential for cytotoxicity in human CTLs. Exp Hematol. 2009; 37:266-75.
14. Shin J, Monti S, Aires D J, Duvic M, Golub T, Jones D A, et al. Lesional gene expression profiling in cutaneous T-cell lymphoma reveals natural clusters associated with disease outcome. Blood. 2007; 110:3015-27.
15. Kuester D, Lippert H, Roessner A, Krueger S. The cathepsin family and their role in colorectal cancer. Pathol Res Pract. 2008; 204:491-500.
16. Kirchgessner H, Dietrich J, Scherer J, Isomäki P, Korinek V, Hilgert I, et al. The transmembrane adaptor protein TRIM regulates T cell receptor (TCR) expression and TCR-mediated signaling via an association with the TCR zeta chain. J Exp Med. 2001; 193:1269-84.
17. Valk E, Leung R, Kang H, Kaneko K, Rudd C E, Schneider H. T cell receptor-interacting molecule acts as a chaperone to modulate surface expression of the CTLA-4 coreceptor. Immunity. 2006; 25:807-21.
18. Boćko D, Bilińska M, Dobosz T, Zoledziewska M, Suwalska K, Tutak A, et al. Lack of association between an exon 1 CTLA-4 gene polymorphism A (49) G and multiple sclerosis in a Polish population of the Lower Silesia region. Arch Immunol Ther Exp. 2003; 51:201-5.
19. Lanier LL. NKG2D Receptor and Its Ligands in Host Defense. Cancer Immunol Res. 2015; 3:575-82.
20. Iqbal J, Weisenburger D D, Greiner T C, Vose J M, McKeithan T, Kucuk C, et al. Molecular signatures to improve diagnosis in peripheral T-cell lymphoma and prognostication in angioimmunoblastic T-cell lymphoma. Blood. 2010; 115:1026-36.
21. Vieyra-Garcia P, Crouch J D, O'Malley J T, Seger E W, Yang C H, Teague J E, et al. Benign T cells drive clinical skin inflammation in cutaneous T cell lymphoma. JCI Insight. 2019; 4.
22. Kamarashev J, Burg G, Mingari M C, Kempf W, Hofbauer G, Dummer R. Differential expression of cytotoxic molecules and killer cell inhibitory receptors in CD8+ and CD56+ cutaneous lymphomas. Am J Pathol. 2001; 158:1593-8.
23. Yang D, Chen Q, Hoover D M, Staley P, Tucker K D, Lubkowski J, et al. Many chemokines including CCL20/MIP-3a display antimicrobial activity. J Leukoc Biol. 2003; 74:448-55.
24. Hahtola S, Tuomela S, Elo L, Häkkinen T, Karenko L, Nedoszytko B, et al. Th1 response and cytotoxicity genes are down-regulated in cutaneous T-cell lymphoma. Clin Cancer Res. 2006; 12:4812-21.
25. Rindler K, Jonak C, Alkon N, Thaler F M, Kurz H, Shaw L E, et al. Single-cell RNA sequencing reveals markers of disease progression in primary cutaneous T-cell lymphoma. Mol Cancer. 2021; 20:124.
26. Jonak C, Alkon N, Rindler K, Rojahn T B, Shaw L E, Porkert S, et al. Single-cell RNA sequencing profiling in a patient with discordant primary cutaneous B-cell and T-cell lymphoma reveals micromilieu-driven immune skewing. Br J Dermatol. 2021; 185:1013-25.
27. Visentini M, Carbonari M, Ghia E, De Propriis S, Guarini A, Girmenia C, et al. A lymphotactin-producing monoclonal T-cell lymphoproliferative disorder with extreme lymphocytopenia and progressive leukoencephalopathy. Leuk Lymphoma. 2006; 47:1421-3.
28. Cairns C M, Gordon J R, Li F, Baca-Estrada M E, Moyana T, Xiang J. Lymphotactin expression by engineered myeloma cells drives tumor regression: mediation by CD4+ and CD8+ T cells and neutrophils expressing XCR1 receptor. The Journal of Immunology. 2001; 167:57-65.
29. Mizumoto Y, Hemmi H, Katsuda M, Miyazawa M, Kitahata Y, Miyamoto A, et al. Anticancer effects of chemokine-directed antigen delivery to a cross-presenting dendritic cell subset with immune checkpoint blockade. Br J Cancer. 2020; 122:1185-93.
30. Alpdogan O, Kartan S, Johnson W, Sokol K, Porcu P. Systemic therapy of cutaneous T-cell lymphoma (CTCL). Chin Clin Oncol. 2019; 8:10.
31. Berti E, Tomasini D, Vermeer M H, Meijer C J, Alessi E, Willemze R. Primary cutaneous CD8-positive epidermotropic cytotoxic T cell lymphomas. A distinct clinicopathological entity with an aggressive clinical behavior. Am J Pathol. 1999:155:483-92.
32. Gambaro K, Quinn M C J, Wojnarowicz P M, Arcand S L, de Ladurantaye M, Barrès V, et al. VGLL3 expression is associated with a tumor suppressor phenotype in epithelial ovarian cancer. Mol Oncol. 2013; 7:513-30.
33. Cody N A L, Shen Z, Ripeau J-S, Provencher D M, Mes-Masson A-M, Chevrette M, et al. Characterization of the 3p12.3-pcen region associated with tumor suppression in a novel ovarian cancer cell line model genetically modified by chromosome 3 fragment transfer. Mol Carcinog. 2009; 48:1077-92.
34. Zhang L, Li L, Mao Y, Hua D. VGLL3 is a prognostic biomarker and correlated with clinical pathologic features and immune infiltrates in stomach adenocarcinoma. Sci Rep. 2020; 10:1355.
35. Hori N, Okada K, Takakura Y, Takano H, Yamaguchi N, Yamaguchi N. Vestigial-like family member 3 (VGLL3), a cofactor for TEAD transcription factors, promotes cancer cell proliferation by activating the Hippo pathway. J Biol Chem. 2020; 295:8798-807.
36. Richmond J, Tuzova M, Parks A, Adams N, Martin E, Tawa M, et al. Interleukin-16 as a marker of Sezary syndrome onset and stage. J Clin Immunol. 2011:31:39-50.
37. Richmond J, Finkel M, Studwell A, Little F, Cruikshank W. Introduction of pro-interleukin-16 inhibits T-lymphoblastic leukemia growth in mice. J Cancer Res Clin Oncol. 2011; 137:1581-5.
38. Querfeld C, Guitart J, Kuzel T M, Rosen S T. Primary cutaneous lymphomas: a review with current treatment options. Blood Rev. 2003; 17:131-42.
39. Gormley R H, Hess S D, Anand D, Junkins-Hopkins J, Rook A H, Kim E J. Primary cutaneous aggressive epidermotropic CD8+ T-cell lymphoma. J Am Acad Dermatol. 2010; 62:300-7.
40. Pulitzer M. Cutaneous T-cell Lymphoma. Clin Lab Med. 2017; 37(3):527-546.
41. Miteva M, El Shabrawi-Caelen L, Fink-Puches R, et al. Alopecia universalis associated with cutaneous T cell lymphoma. Dermatology. 2014: 229(2):65-69.
42. Willemze R, Jansen P M, Cerroni L, et al. Subcutaneous panniculitis-like T-cell lymphoma: definition, classification, and prognostic factors: an EORTC Cutaneous Lymphoma Group Study of 83 cases. Blood. 2008; 111(2):838-845.
43. Jawed, S. I., Myskowski, P. L., Horwitz, S., Moskowitz, A. & Querfeld, C. Primary cutaneous T-cell lymphoma (mycosis fungoides and Sézary syndrome): part II. Prognosis, management, and future directions. J. Am. Acad. Dermatol. 70, 223.e1-17; quiz 240-2 (2014).
44. Smith, B. D. & Wilson, L. D. Management of mycosis fungoides. Part 1. Diagnosis, staging, and prognosis. Oncology 17, 1281-1288 (2003).
45. Redmond, W. J. & Rahbari, H. Mycosis fungoides: a retrospective study. J. Am. Acad. Dermatol. 1, 431-436 (1979).
46. Criscione, V. D. & Weinstock, M. A. Incidence of Cutaneous T-Cell Lymphoma in the United States, 1973-2002. Arch. Dermatol. 143, 854-859 (2007).
47. Wilson, L. D., Hinds, G. A. & Yu, J. B. Age, race, sex, stage, and incidence of cutaneous lymphoma. Clin. Lymphoma Myeloma Leuk. 12, 291-296 (2012).
48. Balagula, Y., Dusza, S. W., Zampella, J., Sweren, R. & Hinds, G. A. Early-onset mycosis fungoides among African American women: a single-institution study. J. Am. Acad. Dermatol. 71, 597-598 (2014).
49. Hinds, G. A. & Heald, P. Cutaneous T-cell lymphoma in skin of color. J. Am. Acad. Dermatol. 60, 359-75; quiz. 376-8 (2009).
50. Imam, M. H., Shenoy, P. J., Flowers, C. R., Phillips, A. & Lechowicz, M. J. Incidence and survival patterns of cutaneous T-cell lymphomas in the United States. Leuk. Lymphoma 54, 752-759 (2013).
51. Balagula, Y., Dusza, S. W., Zampella, J., Sweren, R. & Hinds, G. A. Early-onset mycosis fungoides among young African American women: A single-institution study. in JOURNAL OF INVESTIGATIVE DERMATOLOGY vol. 134 2059-2059.
52. Zampella, J. G. & Hinds, G. A. Racial differences in mycosis fungoides: a retrospective study with a focus on eosinophilia. J. Am. Acad. Dermatol. 68, 967-971 (2013).
53. Sun, G. et al. Poor prognosis in non-Caucasian patients with early-onset mycosis fungoides. J. Am. Acad. Dermatol. 60, 231-235 (2009).
54. Agi, C., Kuhn, D., Chung, J., Zampella, J. & Hinds, G. Racial differences in the use of extracorporeal photopheresis for mycosis fungoides. J. Dermatolog. Treat. 26, 266-268 (2015).
55. Su, C. et al. Racial disparity in mycosis fungoides: An analysis of 4495 cases from the US National Cancer Database. J. Am. Acad. Dermatol. 77, 497-502.e2 (2017).
56. Pariser and Gram, “Feline cutaneous lymphocytosis: case report and summary of the literature,” Journal of Feline Medicine and Surgery (2014) 16, 758-763
57. Miller, “Classification and clinical features in 88 cases of equine cutaneous lymphoma,” J Vet Diagn Invest. 2015 January; 27(1):86-91
58. Wheat, C. M. & Okoye, G. A. Cutaneous T-Cell Lymphoma. in Clinical Cases in Skin of Color: Medical, Oncological and Hair Disorders, and Cosmetic Dermatology (eds. Love, P. B. & Kundu, R. V.) 111-124 (Springer International Publishing, 2016). doi:10.1007/978-3-319-23615-5_10.
59. Egbeto I A, C J Garelli, C Piedra-Mora, N B Wong, C N David, N A Robinson & J M Richmond. “Case Series: Gene Expression Analysis in Canine Vogt-Koyanagi-Harada/Uveodermatologic Syndrome and Vitiligo Reveals Conserved Immunopathogenesis Pathways Between Dog and Human Autoimmune Pigmentary Disorders.” Front Immunol. 2020; 11, doi: 10.3389/fimmu.2020.590558.
60. Garelli C J, N B Wong, C Piedra-Mora, L M Wrijil, G Scarglia, C N David, R M Almela, N A Robinson & J M Richmond. “Shared inflammatory and skin-specific gene signatures reveal common drivers of discoid lupus erythematosus in canines, humans and mice.” Curr Res Immunol. 2021; (2) 41-51, doi.org/10.1016/j.crimmu.2021.03.003
61. Wex T, Bühling F, Wex H, Günther D, Malfertheiner P, Weber E, Brömme D. Human cathepsin W, a cysteine protease predominantly expressed in NK cells, is mainly localized in the endoplasmic reticulum. J Immunol. 2001: 167(4):2172-8.
62. Stoeckle C, Gouttefangeas C, Hammer M, Weber E, Melms A, Tolosa E. Cathepsin W expressed exclusively in CD8+ T cells and NK cells, is secreted during target cell killing but is not essential for cytotoxicity in human CTLs. Exp Hematol. 2009; 37(2):266-75.
63. Kuester D, Lippert H, Roessner A, Krueger S. The cathepsin family and their role in colorectal cancer. Pathol Res Pract. 2008; 204(7):491-500.
64. Brachelente C, Affolter V K, Fondati A, Porcellato I, Sforna M, Lepri E, et al. CD3 and CD20 Coexpression in a Case of Canine Cutaneous Epitheliotropic T-Cell Lymphoma (Mycosis Fungoides). Vet Pathol. 2016; 53:563-6.
65. Meuten D J. Tumors in Domestic Animals. Wiley & Sons, Limited, John; 2016.
66. Gross T L. Skin diseases of the dog and cat: clinical and histopathologic diagnosis. Ames, Iowa: Blackwell Science; 2005.

### OTHER EMBODIMENTS

It is to be understood that while the invention has been described in conjunction with the detailed description thereof, the foregoing description is intended to illustrate and not limit the scope of the invention, which is defined by the scope of the appended claims. Other aspects, advantages, and modifications are within the scope of the following claims.

1. A method comprising:
obtaining a sample comprising skin from a subject, and
determining a level of expression of one, two, three, or all four of cathepsin W (CTSW), T Cell Receptor Associated Transmembrane Adaptor 1 (TRAT1), killer cell lectin like receptor K1 (KLRK1; also known as NKG2D), and/or XCL1/XCL2 (lymphotactin) in the sample.

2. A method of diagnosing a subject as having cutaneous T cell lymphoma, the method comprising:
obtaining a sample comprising skin from a subject;
determining a level of expression of one, two, three, or all four of cathepsin W (CTSW), T Cell Receptor Associated Transmembrane Adaptor 1 (TRAT1), and killer cell lectin like receptor K1 (KLRK1), and/or XCL1/XCL2 (lymphotactin) in the sample;
comparing the level of expression of CTSW, TRAT1, and/or KLRK1 in the sample to a reference level, and identifying the subject as having CTCL wherein a level of expression of CTSW, TRAT1, and/or KLRK1 above the reference level indicates that the subject.

3. The method of claim 1, wherein the sample comprises skin from a lesion, optionally a nodule, tumor, plaque, ulcerated, or rashy patch of skin that is suspected of being CTCL.

4. The method of claim 1, wherein the subject is a mammal.

5. The method of claim 4, wherein the mammal is a human, optionally a human subject having skin of color.

6. The method of claim 5, wherein the subject has Fitzpatrick phototype IV, V, or VI skin.

7. The method of claim 4, wherein the subject is a canine, feline, or equine.

8. The method of claim 2, further comprising administering a treatment for CTCL to a subject identified as having CTCL.

9. The method of claim 8, wherein the treatment comprises a skin-directed therapy, optionally topical steroids, involved-site radiation therapy (RT), total skin electron-beam therapy (TSEBT), or phototherapy/photopheresis, or systemic therapies, optionally immunotherapies (retinoids, interferon, chimeric antigen receptor T cells, or forodesine), small molecules (histone deacetylase inhibitors), antibodies (optionally anti-CCR4, anti-programmed cell death protein 1, anti-CD47, or anti-CD30), chemotherapy, or allogeneic haematopoietic stem cell transplantation.

10. A kit comprising reagents for detecting the biomarkers comprising or consisting of two, three, or all four of CTSW, KLRK1, TRAT1 and/or XCL1/XCL2, and optionally one or more controls.

11. The kit of claim 10, wherein the reagents comprise probes or primers that bind to the two, three, or all four biomarkers and optionally one or more controls.

12. The kit of claim 10, wherein the controls are housekeeping genes, optionally one, two, three, four, or all five of B2M, RPL13A, CCZ1, HPRT1, and/or GAPDH.

13. The kit of claim 10, which comprises a microarray.

14. The kit of claim 10, wherein the probes or primers bind to at least 20 consecutive nucleotides of a biomarker sequence shown in SEQ ID NO:7-10, or a human homolog thereof.


SEQUENCE LISTING (15 sequences)

SEQ ID NO: 1 - DNA, 20 bp, synthetic construct
  source 1..20: mol_type = other DNA
gcttcctgctacccacactt

SEQ ID NO: 2 - DNA, 20 bp, synthetic construct
  source 1..20: mol_type = other DNA
aagatgtccagacgacgagc

SEQ ID NO: 3 - DNA, 20 bp, synthetic construct
  source 1..20: mol_type = other DNA
acccattcttggggaacacc

SEQ ID NO: 4 - DNA, 20 bp, synthetic construct
  source 1..20: mol_type = other DNA
aacccacaagcaggacagag

SEQ ID NO: 5 - DNA, 19 bp, synthetic construct
  source 1..19: mol_type = other DNA
gatgggcgtgaaccatgag

SEQ ID NO: 6 - DNA, 19 bp, synthetic construct
  source 1..19: mol_type = other DNA
tcatgaggccctccacgat

SEQ ID NO: 7 - DNA, 100 bp, synthetic construct
  source 1..100: mol_type = other DNA
ccagccactggagctgaaacaggtctttgcgttgttccagatccagtacaaccggagttactcaaacccagaagaatatgctcgtcgtctggacatcttt

SEQ ID NO: 8 - DNA, 100 bp, synthetic construct
  source 1..100: mol_type = other DNA
gtacagttactctgaagactatattcccagggaggaggagtattatgttgaagacacaccaatttatggtaacttagatgacatggtcccagaaccagtg

SEQ ID NO: 9 - DNA, 100 bp, synthetic construct
  source 1..100: mol_type = other DNA
ccttaattataactctgaacctgtaacacatggtacttctccacgaaggcaaaagagaaagcctacattaatcaccagcaaatgtggagaaaactcatct

SEQ ID NO: 10 - DNA, 100 bp, synthetic construct
  source 1..100: mol_type = other DNA
tttctcactgaattcatcgtggaaggtgtggggagtgaagtcctagaaaggagcatctgtatgagtctgacgaccaagagactgccagttaaaagcatca

SEQ ID NO: 11 - DNA, 100 bp, synthetic construct
  source 1..100: mol_type = other DNA
tcttctggtccacactgaattcactcccaatgagcaggatgagtttagctgccgtgtaaagcatgttactctcagtgagccccagatcgttaagtgggac

SEQ ID NO: 12 - DNA, 100 bp, synthetic construct
  source 1..100: mol_type = other DNA
ggccggaaggttgtagtcgtgcgctgtgaaggcatcaacatttctggcaatttctacagaaacaagttgaagtacctggccttcctccgcaagcggatga

SEQ ID NO: 13 - DNA, 100 bp, synthetic construct
  source 1..100: mol_type = other DNA
gccgagaaaagtacaattcacatgaggaaaacacctagtgtgtcacttacatctgtgcatccggacttaatgaagattctgggtgacatcaacagtgatt

SEQ ID NO: 14 - DNA, 100 bp, synthetic construct
  source 1..100: mol_type = other DNA
ccatcacatcgtagccctctgtgtgctcaagggaggctataaattctttgctgacctgctggattatatcaaagcactgaacagaaatagtgatggatcc

SEQ ID NO: 15 - DNA, 100 bp, synthetic construct
  source 1..100: mol_type = other DNA
cagtgacacccactcttccaccttcgacgccggggctggcattgccctcaatgaccactttgtcaagctcatttcctggtatgacaatgaatttggctac